(19) United States
(12) Patent Application Publication
(10) Pub. No.: US 2003/0143604 A1
(43) Pub. Date: Jul. 31, 2003

(54) Real-time monitoring of PCR amplification using nanoparticle probes

(76) Inventors: James J. Storhoff, Evanston, IL (US); Brett Fritz, Chicago, IL (US); Mark Herrmann, Clinton, UT (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/306,630

(22) Filed: Nov. 27, 2002

Related U.S. Application Data
(60) Provisional application No. 60/334,644, filed on Nov. 30, 2001.

(51) Int. Cl. C12Q 1/68; C12P 19/34

(52) U.S. Cl. 435/6; 435/91.2

(57) ABSTRACT

The present invention relates to the use of nanoparticle detection probes to monitor amplification reactions, especially polymerase chain reactions (“PCR”). More specifically, the present invention involves the use of nanoparticles oligonucleotide conjugates treated with a protective agent such as bovine serum albumin in an homogeneous assay format in order to quantitatively and qualitatively detect a target polynucleotide.

### CROSS-REFERENCE

This application claims the benefit of U.S. Provisional application No. 60/334,644, filed Nov. 30, 2001, which is incorporated by reference in its entirety.

### FIELD OF THE INVENTION

This invention relates to a method, composition, and kit for determining the presence of a target polynucleotide in a sample. In particular, this invention relates to a method for determining the presence of a target polynucleotide by real-time monitoring of an amplification reaction, preferably the polymerase chain reaction (PCR) using passivated nanoparticle probes. This invention also relates to methods for performing nucleic acid amplification, preferably the polymerase chain reaction (PCR), in the presence of passivated nanoparticle probes.

### BACKGROUND OF THE INVENTION

The sensitive detection of nucleic acids in a clinical sample opened a new era in the diagnosis of infectious diseases and other fields. Powerful nucleic acid amplification and detection methods are available which allow the detection of very small copy numbers of target polynucleotides. Tremendous progress has been made concerning the qualitative detection of nucleic acids, but the quantitative detection is still a challenge for the existing methods, especially for amplification methods based on the exponential amplification of a target polynucleotide. The best known amplification method of this type is the polymerase chain reaction (PCR). U.S. Pat No. 4,683,195; U.S. Pat No. 4,683,202.

Nucleic acids in a sample are usually first amplified by the amplification method and subsequently detected by the detection method. This sequential approach is based on a single end-point measurement after the amplification reaction is completed. The amount of amplified product observed at the end of the reaction is very sensitive to slight variations in reaction components because the amplification reaction is typically exponential. Therefore, the accuracy and precision of quantitative analysis using endpoint measurements is poor. Furthermore, endpoint measurements can produce a hook effect whereby high concentrations of a target polynucleotide to be amplified yield inaccurately low values.

In contrast to end-point determinations of amplified polynucleotides, real-time monitoring of amplification reaction product generation offers the possibility of better precision and accuracy in quantitative measurements because the measurements are taken during the exponential phase of the amplification process. In contrast to classical end-point measurements, multiple measurements are taken during real-time monitoring. During the exponential phase of the amplification process, none of the reaction components are limiting, and therefore the affect on accuracy of reaching a maximum signal are eliminated. Real-time monitoring of PCR is based on kinetic measurements offering a better and a more complete picture of the PCR process. A number of real-time monitoring methods have been developed, however the methods use fluorescent signals in all cases. Although the fluorescence signaling methodology has been quite successful, it may be improved by: (a) enhancing the specificity of the signaling probe since molecular fluorophore labels exhibit broad melting transitions, (b) enhancing the sensitivity of the labels used for detection, and (c) developing a signaling system that utilizes lower cost instrumentation and reagents used to perform the real time assay. This limits the earliest possible detection of amplifying DNA (RNA) because of the presence of unquenched or background fluorescence. See Heid et al., (1996) Genome Res., Vol. 6(10), pp. 986-994.

There remains a need for an assay method that utilizes an amplification reaction and that can be used for highly specific and sensitive qualitative and quantitative measurements of a target polynucleotide with low cost instrumentation. More specifically there remains a need for an assay method with better specificity than fluorescence labels which will result in higher precision and accuracy in nucleic acid testing, as well as more cost effective reagents and instrumentation. To accomplish this, there remains a need for a labeling technology that exhibits higher specificity than molecular fluorophore labels or intercalator dyes that can be monitored with simple instrumentation and with rapid incubation and signal generation time to allow the real-time monitoring of an amplification reaction. Finally, there remains a need for an assay which can measure a target polynucleotide in an amplification reaction without a high-dose hook effect.

The present invention relates to the use of nanoparticles as the detection technology to monitor amplification reactions such as the polymerase chain reaction (“PCR”), in an all-in-one-tube format. More specifically, the present invention involves the use of passivated nanoparticles to measure the kinetics of a PCR reaction in an all-in-one assay format in order to quantitatively and qualitatively detect a target polynucleotide. The invention has the advantages of a robust, highly specific detection probe coupled with rapid signal generation to allow multiple measurements to be taken during the linear phase of a PCR reaction with simple, cost effective spectrophotometric detection. The enhanced specificity of the nanoparticle probes enables probe/target hybridization and probe detection under extremely stringent conditions which leads to accurate identification of nucleic acid sequences. This provides a more complete picture of the amplification process and sensitive qualitative and quantitative detection of nucleic acids with improved precision and accuracy.

Nanoparticles have been a subject of intense interest owing to their unique physical and chemical properties which stem from their size. Due to these properties, nanoparticles offer a promising pathway for the development of new types of biological sensors that are more sensitive, more specific, and more cost effective than conventional detection methods. Methods for synthesizing nanoparticles and methodologies for studying their resulting properties have been widely developed over the past 10 years (Klabunde, editor, Nanoscale Materials in Chemistry, WileyInterscience, 2001). However, their use in biological sensing has been limited by the lack of robust methods for functionalizing nanoparticles with biological molecules of interest due to the inherent incompatibilities of these two disparate materials. A highly effective method for functionalizing nanoparticles with modified oligonucleotides has been developed. See U.S. Pat. Nos. 6,361,944 and 6,417,340 (assignee: Nanosphere, Inc.), which are incorporated by reference in their entirety. The process leads to nanoparticles that are heavily functionalized with oligonucleotides which have surprising particle stability and hybridization properties. The resulting DNA-modified particles have also proven to be very robust as evidenced by their stability in solutions containing elevated electrolyte concentrations, stability towards centrifugation or freezing, and thermal stability when repeatedly heated and cooled. This loading process also is controllable and adaptable. Nanoparticles of differing size and composition have been functionalized, and the loading of oligonucleotide recognition sequences onto the nanoparticle can be controlled via the loading process.

The aforementioned loading method for preparing DNA-modified nanoparticles, particularly DNA-modified gold nanoparticle probes, has led to the development of a new colorimetric sensing scheme for oligonucleotides. This method is based on the hybridization of two gold nanoparticle probes to two distinct regions of a DNA target of interest. Since each of the probes are functionalized with multiple oligonucleotides bearing the same sequence, the binding of the target results in the formation of target DNA/gold nanoparticle probe aggregate when sufficient target is present. The DNA target recognition results in a red to purple/blue colorimetric transition due to the decrease in interparticle distance of the particles. This colorimetric change can be monitored optically, with a UV-vis spectrophotometer, or visually with the naked eye. In addition, the color is intensified when the solutions are concentrated onto a membrane. Therefore, a simple red to blue colorimetric transition provides evidence for the presence or absence of a specific DNA sequence. Using this assay, femtomole quantities and nanomolar concentrations of model DNA targets and polymerase chain reaction (PCR) amplified nucleic acid sequences have been detected. Importantly, it has been demonstrated that gold probe/DNA target complexes exhibit extremely sharp melting transitions which makes them highly specific labels for DNA targets. In a model system, one base insertions, deletions, or mismatches were easily detectable via the spot test based on color and temperature, or by monitoring the melting transitions of the aggregates spectrophotometrically (Storhoff et. al, J. Am. Chem. Soc.,120, 1959 (1998.). Due to the sharp melting transitions, the perfectly matched target could be detected even in the presence of the mismatched targets when the hybridization and detection was performed under extremely high stringency (e.g., a single degree below the melting temperature of the perfect probe/target match). It is important to note that with broader melting transitions such as those observed with molecular fluorophore labels, hybridization and detection at a temperature close to the melting temperature would result in significant loss of signal due to partial melting of the probe/target complex leading to lower sensitivity, and also partial hybridization of the mismatched probe/target complexes leading to lower specificity due to mismatched probe signal. Therefore, nanoparticle probes offer higher specificity detection for nucleic acid detection methods such as real time detection.

A variety of methods have been developed for single nucleotide polymorphism (SNP) detection and are commercially available (Kwok, P. Y., Annu. Rev. Genomics Hum. Genet., 2, 235, (2001). For the research market, the most widely used instruments are based on real time fluoresecence detection methods. Detection monitoring in real time provides the end user with more reliable information and extends the capabilities of a given system, while decreasing the amount of time associated with performing the assay.

The Applicants have developed a real time PCR amplification detection system using nanoparticle-oligonucleotide conjugates as detection probes and demonstrate that PCR amplification can occur in the presence of the nanoparticle probes, and that PCR amplified targets may be detected with nanoparticle probes either spectrophotometrically or by spotting the probe/target complex onto a membrane. The method and system of the present invention eliminates the need for adding the nanoparticle probes post-PCR, ultimately simplifying any assay designed around PCR amplification and nanoparticle probes, and also allow monitoring of nanoparticle probe hybridization in real time, based on colorimetric changes that occur in solution.

### SUMMARY OF THE INVENTION

The current invention relates to the use of nanoparticle technology to monitor amplification reactions, especially polymerase chain reactions (“PCR”). More specifically, the current invention involves the use of passivated nanoparticle probes to measure the kinetics of a PCR reaction in an all-in-one assay format in order to quantitatively and qualitatively detect a target polynucleotide.

One embodiment of the invention is directed to a method for detecting the presence of a target polynucleotide in a sample comprising: (A) providing a reaction and detection mixture comprising in combination: (1) a sample; (2) a nucleic acid amplification system; and (3) a nanoparticle detection system comprising a passivated nanoparticle conjugate capable of binding to the amplified target nucleic acid; (B) amplifying said target polynucleotide through at least one amplification cycle; (C) allowing the binding of said nanoparticle probe to said amplified target polynucleotide; optionally repeating steps B and C; and (D) detecting the presence of said target polynucleotide by observing a detectable changes determined after at least one amplification cycle.

In another embodiment of the invention, the target polynucleotide comprises first and second complimentary strands; and the nucleic acid amplification system comprises: (1) a thermostable DNA polymerase; (2) 2′ deoxynucleoside-5′-triphosphates; (3) a forward-primer capable of binding to the first complimentary strand; and (4) a reverse-primer capable of binding to the second complimentary strand in a position that will direct DNA synthesis toward the site of annealing of the forward-priming oligonucleotide. The amplification system preferably utilizes the polymerase amplification reaction. If desired, thermal labile antibody against the thermal stable DNA polymerase may be used in a “hot start” amplification reaction.

In a further embodiment of the invention, a method for quantifying the amount of target polynucleotide in a sample is provided. The amount of signal produced is related to the amount of target polynucleotide in the sample. The signal determinations are made during an exponential phase of the amplification process and involve (a) determining a threshold cycle number at which the signal generated from amplification of the target polynucleotide in a sample reaches a fixed threshold value above a baseline value; and (b) calculating the quantity of the target polynucleotide in the sample by comparing the threshold cycle number determined for the target polynucleotide in a sample with the threshold cycle number determined for target polynucleotides of known amounts in standard solutions.

In yet another embodiment of the invention, a method is provided for detecting the presence of a target polynucleotide in a sample, the target polynucleotide comprising a first and a second complimentary strand. The method comprises (a) providing a reaction and detection mixture comprising in combination: (1) a sample, (2) a thermostable DNA polymerase, (3) 2′ deoxynucleoside-5′-triphosphates, (4) a forward-primer capable of binding to the first complimentary strand, (5) a reverse-primer capable of binding to the second complimentary strand in a position that will direct DNA synthesis toward the site of annealing of the forward-priming oligonucleotide, and (6) a nanoparticle detection probe system comprising a passivated nanoparticle having oligonucleotides bound thereto, the nanoparticle capable of binding to the amplified target nucleic acid; (b) denaturing said target polynucleotide for an initial denaturation period; (c) denaturing said target polynucleotide for a cycle denaturation period; (d) incubating the reaction and detection mixture to allow binding of said nanoparticle probe to said amplified target polynucleotide; (e) determining the amount of signal generated by the nanoparticle probe; (g) annealing said forward priming and reverse priming oligonucleotides to the target polynucleotide; (h) synthesizing polynucleotide strands complementary to said first and second complementary strands of said target polynucleotide, said synthesis being catalyzed by the thermostable DNA polymerase; (i) optionally repeating steps (c)-(h); and (j) detecting the presence of said target polynucleotide by analyzing the amount of signal generated after at least one amplification cycle.

In yet another embodiment of the invention, a method is provided for detecting the presence of a target polynucleotide in a sample, the target polynucleotide comprising a first and a second complimentary strand. The method comprises (a) providing a reaction and detection mixture comprising in combination: (1) a sample, (2) a thermostable DNA polymerase, (3) 2′ deoxynucleoside-5′-triphosphates, (4) a forward-primer composed of a passivated nanoparticle probe with attached DNA primer sequence capable of binding to the first complimentary strand, (5) a reverse-primer capable of binding to the second complimentary strand in a position that will direct DNA synthesis toward the site of annealing of the forward-priming oligonucleotide attached to the nucleotide, and (6) a nanoparticle detection probe system comprising one or more types of nanoparticles having one or more types of oligonucleotides bound thereto, the oligonucleotides bound to the nanoparticles have a sequence that is complementary to at least a portion of the sequence of the extension product of the nanoparticle labeled DNA primer sequence; and

(b) denaturing said target polynucleotide for an initial denaturation period; (c) denaturing said target polynucleotide for a cycle denaturation period; (d) incubating the reaction and detection mixture to allow binding of said nanoparticle probe to said amplified target polynucleotide attached to the passivated nanoparticle probe; (e) determining the amount of signal generated by the nanoparticle probe; (g) annealing said forward priming and reverse priming oligonucleotides to the target polynucleotide; (h) synthesizing polynucleotide strands complementary to said first and second complementary strands of said target polynucleotide, said synthesis being catalyzed by the thermostable DNA polymerase; (i) optionally repeating steps (c)-(h); and (j) detecting the presence of said target polynucleotide by analyzing the amount of signal generated after at least one amplification cycle.

In yet another embodiment of the invention, a method is provided for detecting the presence of a target polynucleotide in a sample, the target polynucleotide comprising a first and a second complimentary strand. The method comprises (a) providing a reaction and detection mixture comprising in combination: (1) a sample, (2) a thermostable DNA polymerase, (3) 2′ deoxynucleoside-5′-triphosphates, (4) a forward-primer capable of binding to the first complimentary strand, (5) a reverse-primer composed of a passivated nanoparticle probe with attached DNA primer sequence capable of binding to the second complimentary strand in a position that will direct DNA synthesis toward the site of annealing of the forward-priming oligonucleotide attached to the nucleotide, and (6) a nanoparticle detection probe system comprising one or more types of nanoparticles having one or more types of oligonucleotides bound thereto, the oligonucleotides bound to the nanoparticles have a sequence that is complementary to at least a portion of the sequence of the extension product of the nanoparticle labeled DNA primer sequence; and

(b) denaturing said target polynucleotide for an initial denaturation period; (c) denaturing said target polynucleotide for a cycle denaturation period; (d) incubating the reaction and detection mixture to allow binding of said nanoparticle probe to said amplified target polynucleotide attached to the passivated nanoparticle probe; (e) determining the amount of signal generated by the nanoparticle probe; (g) annealing said forward priming and reverse priming oligonucleotides to the target polynucleotide; (h) synthesizing polynucleotide strands complementary to said first and second complementary strands of said target polynucleotide, said synthesis being catalyzed by the thermostable DNA polymerase; (i) optionally repeating steps (c)-(h); and (j) detecting the presence of said target polynucleotide by analyzing the amount of signal generated after at least one amplification cycle.

In yet another embodiment of the invention, a method is provided for detecting the presence of a target polynucleotide in a sample, the target polynucleotide comprising a first and a second complimentary strand. The method comprises (a) providing a reaction and detection mixture comprising in combination: (1) a sample, (2) a thermostable DNA polymerase, (3) 2′ deoxynucleoside-5′-triphosphates, (4) a forward-primer composed of a passivated nanoparticle probe with attached DNA primer sequence capable of binding to the first complimentary strand, and (5) a reverse-primer composed of a passivated nanoparticle probe with attached DNA primer sequence capable of binding to the second complimentary strand in a position that will direct DNA synthesis toward the site of annealing of the forward-priming oligonucleotide attached to the nucleotide;

(b) denaturing said target polynucleotide for an initial denaturation period; (c) denaturing said target polynucleotide for a cycle denaturation period; (d) incubating the reaction mixture at a temperature to allow hybridization of the passivated nanoparticle probes containing the amplified target regions; (e) determining the amount of signal generated by the nanoparticle probe; (g) annealing said forward priming and reverse priming oligonucleotides to the target polynucleotide; (h) synthesizing polynucleotide strands complementary to said first and second complementary strands of said target polynucleotide, said synthesis being catalyzed by the thermostable DNA polymerase; (i) optionally repeating steps (c)-(h); and (j) detecting the presence of said target polynucleotide by analyzing the amount of signal generated after at least one amplification cycle.

These and other embodiments of the invention will become apparent in light of the detailed description below.

### DESCRIPTION OF THE DRAWINGS

FIG. 1: Part A is a schematic diagram illustrating real time detection of nucleic acid amplification using gold nanoparticle probes. In step 1, the nucleic acid target is denatured in a solution containing the gold nanoparticle probes and primers. In step 2, the gold nanoparticle probes and primers are bound to the nucleic acid target, and the optical signal from the gold nanoparticle probes is measured. In step 3, a copy of the DNA sequence is generated from the primers via DNA polymerase resulting in amplification of the number of nucleic acid targets. Steps 1-3 are repeated until measurable optical signal is generated from the gold nanoparticle probes. Part B is a schematic diagram illustrating nucleic acid amplification and detection using gold nanoparticle probe primers. In step 1, the nucleic acid target is denatured in the presence of the gold nanoparticles with attached primers. In step 2, the gold nanoparticles with attached primers are hybridized to the nucleic acid target, and a copy of the complementary DNA sequence is generated from the nucleic acid primers attached to the nanoparticles. Steps 1 and 2 are repeated, and the optical signal generated from the binding of complementary target amplified nanoparticle probes is measured. These steps may be repeated as necessary to generated detectable optical signal from the nanoparticle probes. Part C is a schematic diagram illustrating real time detection of nucleic acid amplification using a combination of gold nanoparticle primers and gold nanoparticle probes. In step 1, the nucleic acid target is denatured in the presence of the gold nanoparticle probes, gold nanoparticle primers, and the normal primers. In step 2, the gold nanoparticles with attached nucleic acid primer and the reverse nucleic acid primer are hybridized to the nucleic acid target under the appropriate conditions, and a copy of the nucleic acid target is generated from the 3′ end of the primer sequences. Steps 1 and 2 are subsequently repeated and the optical changes associated with binding of the nanoparticle probes with amplified sequence to complementary gold nanoparticle probe are measured.

FIG. 2.: Thermal denaturation analysis of wild type and mutant gold nanoparticle probe sets with complementary nucleic acid targets and targets containing a single base mismatch. Part A illustrates the melting analysis of the wild type APC gene gold probe set (SEQ ID NO: 1 and 3) with wild type (SEQ ID NO: 5) (perfect match) and mutant (SEQ ID NO: 6) (single base mismatch) nucleic acid targets. Part B illustrates the melting analysis of the mutant APC gene gold probe (SEQ ID NO: 2 and 3) with mutant (SEQ ID NO: 6) (perfect match) and wild type (SEQ ID NO: 5) (single base mismatch) nucleic acid targets.

FIG. 3.: Part A is a schematic diagram of the polymerase chain reaction (PCR) process. In step 1, the nucleic acid target is denatured. In step 2, nucleic acid primers hybridize to complementary regions of the nucleic acid target. In step 3, a copy of the nucleic acid sequence is generated from the 3′ end of the nucleic acid primers via a thermostable polymerase (e.g. Taq polymerase). Steps 1-3 are repeated to amplify the number of copies of the desired nucleic acid sequence. Part B is a schematic diagram of the PCR amplification reaction of the methylene tetrahyrdofolate reductase (MTHFR) gene (SEQ ID NO: 4) in the presence of gold nanoparticles with attached nucleic acid sequences specific for the APC gene (SEQ ID NO: 1 and 3). Note in this model system designed to test the efficacy of the PCR process with gold nanoparticles with attached nucleic acids in the reaction mixture, the nucleic acid sequences attached to the gold nanoparticles are not complementary to the target. In step 1, the target is denatured in the presence of the nanoparticle probes and PCR reaction components. In step 2, the primers are bound to the nucleic acid target sequence. In step 3, extension of the primers by Taq polymerase is inhibited by the presence of the gold nanoparticle probes as evidenced by a loss in amplified MTHFR gene PCR product (see FIG. 4 for experimental results). Part C is a schematic diagram of the PCR amplification reaction of the methylene tetrahyrdofolate reductase (MTHFR) gene (SEQ ID NO: 4) in the presence of gold nanoparticles with attached nucleic acid sequences specific for the APC gene (SEQ ID NO: 1 and 3) that have been further passivated with BSA prior to addition to the PCR reaction mixture. The MTHFR gene PCR amplification process is the same as described in FIG. 3B. The MTHFR gene PCR amplification reaction proceeds uninhibited in the presence of the gold nanoparticle probes with the added BSA in solution (see FIG. 5 for experimental results).

FIG. 4: Gel electrophoresis image of the MTHFR gene PCR amplification reaction (SEQ ID NO: 4) with added gold nanoparticle probes (SEQ ID NO: 1 and 3) at concentrations of 400 pM, 2 nM, and 4 nM compared to the same reaction without gold nanoparticle probes. The gold nanoparticle probes inhibit the PCR amplification reaction in a dose dependent manner.

FIG. 5: Gel electrophoresis image of the MTHFR gene PCR amplification reaction (SEQ ID NO: 4) with added gold nanoparticle probes (SEQ ID NO: 1 and 3) that have been further passivated with bovine serum albumin (BSA, final concentration of 0.05%). The gold nanoparticle probes were added to the PCR reaction mixture at concentrations of 360 pM, 1.8 nM, and 3.6 nM and compared to the same reaction without gold nanoparticle probes as a positive control. Additional controls containing added Tris buffer (pH 8) and added BSA without gold nanoparticles also were tested. The BSA passivated gold nanoparticle probes do not interfere with the PCR amplification reaction.

FIG. 6: Spot test of gold nanoparticle probes (SEQ ID NO: 1 and 3) with complementary synthetic APC gene 78 base target 1 (SEQ ID NO:5) with added BSA. The purple spots recorded for the probe/APC gene target solutions (30 nM and 50 nM target) demonstrate that the BSA does not interfere with nucleic acid hybridization on the gold nanoparticle probes and also does not interfere with probe aggregation which leads to the observed color changes.

FIG. 7: A schematic diagram representing the detection of the PCR amplified APC gene sequence (SEQ ID NO:5) with complementary gold nanoparticle probes (SEQ ID NO: 1 and 3) by measuring optical changes in solution (see FIG. 8 for experimental data).

FIG. 8: UV-visible spectrum of 30 nm diameter gold nanoparticle probes (SEQ ID NO: 1 and 3) hybridized to a complementary PCR amplified APC gene sequence (SEQ ID NO:5). A negative control solution that contains the gold nanoparticle probes with no PCR amplified product is shown for comparison. A colorimetric red shift is observed for the gold probe/PCR amplicon solution in the UV-visible spectrum which leads to increased extinction values in the 555-630 nm region and a decrease in extinction below 540 nm. This experiment demonstrates that PCR amplicon/gold nanoparticle probe binding produces optical changes that may be monitored with a spectrophotometer or other types or readers that can detect optical changes.

FIG. 9: Spot test detection assay on nylon performed with wild type (SEQ ID NO: 1 and 3) and mutant (SEQ ID NO: 2 and 3) 30 nm diameter gold nanoparticle probe sets that are hybridized to PCR amplified APC gene targets 1 and 2 (SEQ ID NO: 5 and 6, respectively). The perfectly matched probe/target solutions exhibit a blue color while the single base mismatch target/probe solution exhibit red spots under these hybridization conditions, indicating single base mismatch specificity with the chosen probe sequences.

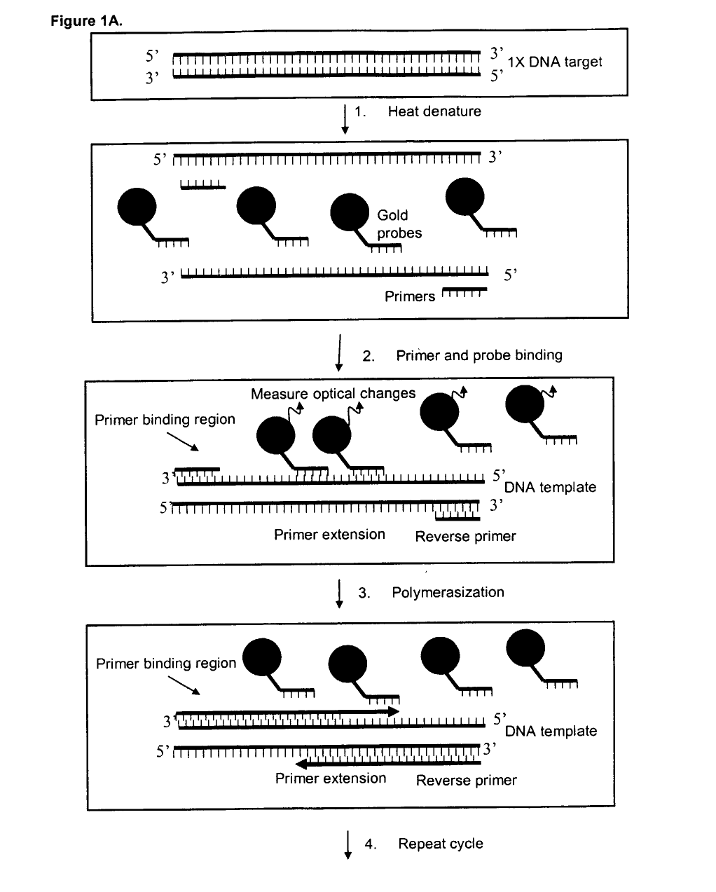

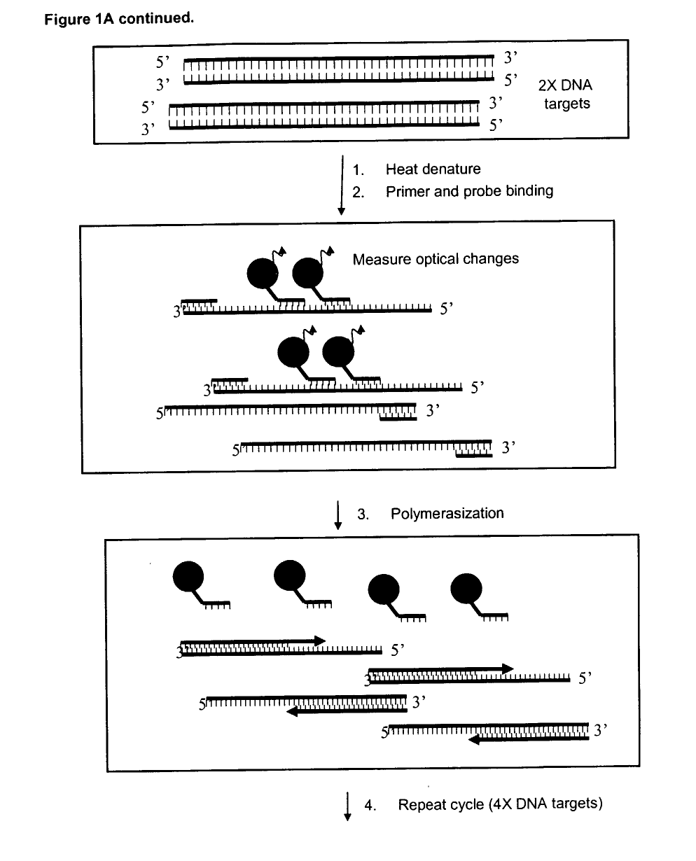

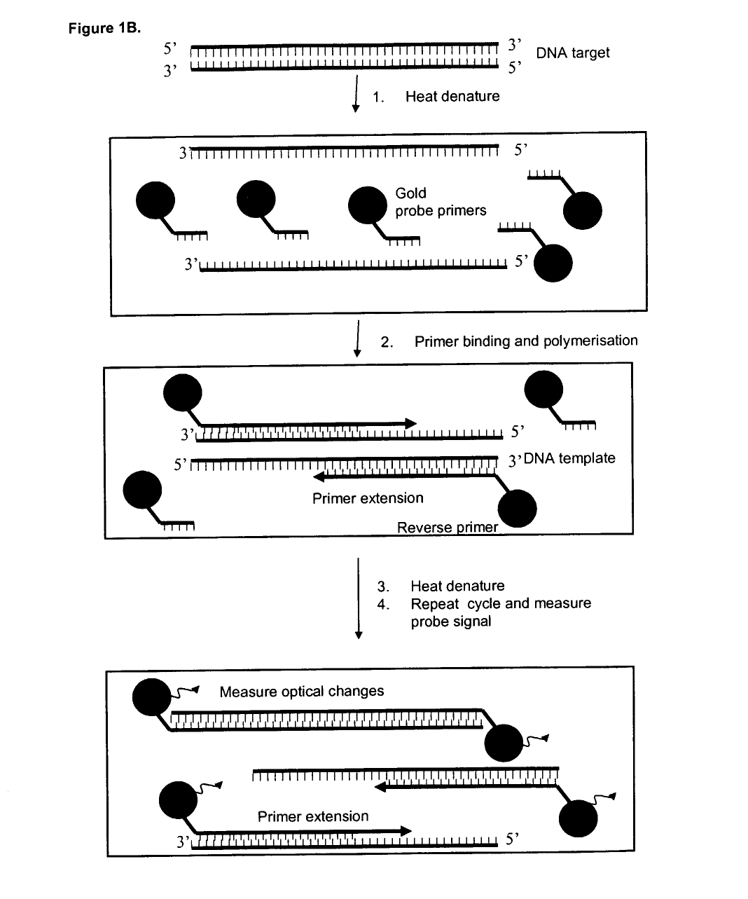

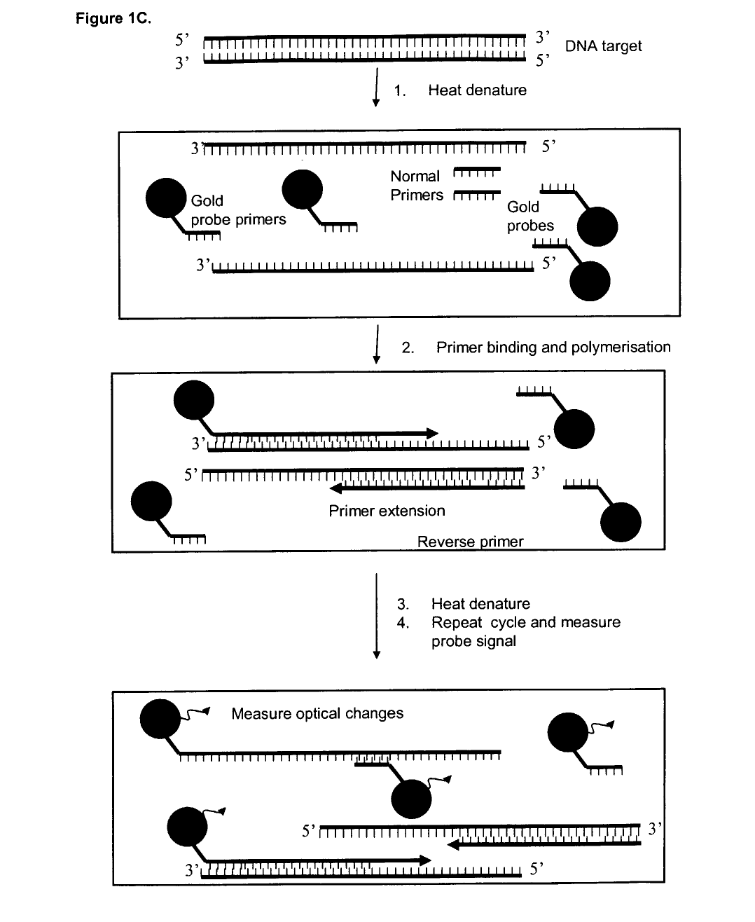

### DETAILED DESCRIPTION OF THE INVENTION

A. Definitions

“Polynucleotide” refers to a compound or composition which is a polymeric nucleotide having in the natural state about 6 to 500,000 or more nucleotides and having in the isolated state about 6 to 50,000 or more nucleotides, usually about 6 to 20,000 nucleotides, more frequently 6 to 10,000 nucleotides. The term “polynucleotide” includes oligonucleotides and nucleic acids from any source in purified or unpurified form, naturally occurring or synthetically produced, including DNA (dsDNA and ssDNA) and RNA, usually DNA, and may be t-RNA, m-RNA, r-RNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and fragments thereof, and the like. The polynucleotide is typically composed of the nucleotides adenosine, guanosine, adenosine, and thymidine. However, the polynucleotide can be composed of other nucleotides, for example de-aza guanosine or preferably inosine, as long as they do not destroy the binding of the polynucleotide to its target.

“Primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, i.e., in the presence of nucleotides and an inducing agent such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and use of the method. For example, for diagnostics applications, depending on the complexity of the target sequence, the oligonucleotide primer typically contains 15-25 or more nucleotides, although it may contain fewer nucleotides.

The primers herein are selected to be “substantially” complementary to the different strands of the target polynucleotide. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to be amplified to hybridize therewith and thereby form a template for synthesis of the extension product of the other primer.

“Target polynucleotide” refers to the polynucleotide in a sample of which at least a portion is intended to be amplified by the amplification reaction. Where an amplification reactions that utilize oligonucleotide primers for an extension reaction are used, such as PCR, the target polynucleotide is that nucleotide to which the extension primers are intended to bind.

“Threshold cycle number” is an amplification cycle number at which point signal intensity reaches or exceeds a certain level.

“Passivating agent” (otherwise referred to “a protective agent”) refers to a substance that will modify covalently or non-covalently at least a portion of surfaces of the nanoparticles that are not bound to oligonucleotides, that will not interfere or substantially interfere with the nucleic acid amplification reaction, and that can withstand heating and cooling steps of the amplification reaction without dissociating or substantially dissociating from the nanoparticle surface. Without being bound by any theory of operation for this invention, it is believed that the passivating agent associates or coats naked nanoparticle surfaces and protects against nucleic acid amplification reaction enzymes or components such as PCR taq polymerase from binding to the naked surfaces and thus adversely affecting the amplification reaction. Suitable, but non-limiting, examples of passivating agents include bovine serum albumin (BSA), casein, streptavidin, polyethylene glycol (PEG), acid terminated and amine terminated thiols such as mercaptourdecanoic acid and mercaptoethylamine, and other small thiol containing peptides such as glutathione.

B. Nanoparticle-Oligonucleotide Probes

Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Taransactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988).

Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky et al., J. Am. Chem. Soc., 112, 9438 (1990); Ushida et al., J. Phys. Chem., 95, 5382 (1992).

Suitable nanoparticles are also commercially available from, e.g., Ted Pella, Inc. (gold), Amersham Corporation (gold) and Nanoprobes, Inc. (gold).

Presently preferred for use in detecting nucleic acids are gold nanoparticles. Gold colloidal particles have high extinction coefficients for the bands that give rise to their beautiful colors. These intense colors change with particle size, concentration, interparticle distance, and extent of aggregation and shape (geometry) of the aggregates, making these materials particularly attractive for colorimetric assays. For instance, hybridization of oligonucleotides attached to gold nanoparticles with oligonucleotides and nucleic acids results in an immediate color change visible to the naked eye. For a description of suitable and preferred nanoparticles, see (see, e.g., U.S. Pat. Nos. 4,683,195 and 4,683,202 as well as published international application nos. PCT/US01/01190, filed Jan. 12, 2001; PCT/US01/10071, filed Mar. 28, 2001; PCT/US01/46418, filed Dec. 7, 2001; and PCT/US01/25237, filed Aug. 10, 2001, which are incorporated by reference in their entirety.

The nanoparticles, the oligonucleotides or both are functionalized in order to attach the oligonucleotides to the nanoparticles. Such methods are known in the art. For instance, oligonucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. Chem. Commun. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

Each nanoparticle will have a plurality of oligonucleotides attached to it. As a result, each nanoparticle-oligonucleotide conjugate can bind to a plurality of oligonucleotides or nucleic acids having the complementary sequence.

Oligonucleotides of defined sequences are used for a variety of purposes in the practice of the invention. Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

The invention provides methods of detecting amplified nucleic acids in a nucleic acid amplification reaction. Any type of amplified nucleic acid may be detected, and the methods may be used, e.g., for the diagnosis of disease and in sequencing of nucleic acids. Examples of nucleic acids that can be detected by the methods of the invention include genes (e.g., a gene associated with a particular disease), viral RNA and DNA, bacterial DNA, fungal DNA, cDNA, mRNA, RNA and DNA fragments, oligonucleotides, synthetic oligonucleotides, modified oligonucleotides, single-stranded and double-stranded nucleic acids, natural and synthetic nucleic acids, etc. Thus, examples of the uses of the methods of detecting nucleic acids include: the diagnosis and/or monitoring of viral diseases (e.g., human immunodeficiency virus, hepatitis viruses, herpes viruses, cytomegalovirus, and Epstein-Barr virus), bacterial diseases (e.g., tuberculosis, Lyme disease, H. pylori, Escherichia coli infections, Legionella infections, Mycoplasma infections, Salmonella infections), sexually transmitted diseases (e.g., gonorrhea), inherited disorders (e.g., cystic fibrosis, Duchene muscular dystrophy, phenylketonuria, sickle cell anemia), and cancers (e.g., genes associated with the development of cancer); in forensics; in DNA sequencing; for paternity testing; for cell line authentication; for monitoring gene therapy; and for many other purposes.

The methods of detecting amplified nucleic acids from nucleic acid amplification reactions based on observing a color change with the naked eye are cheap, fast, simple, robust (the reagents are stable), do not require specialized or expensive equipment, and little or no instrumentation is required. This makes them particularly suitable for use in, e.g., research and analytical laboratories in DNA sequencing, in the field to detect the presence of specific pathogens, in the doctor's office for quick identification of an infection to assist in prescribing a drug for treatment, and in homes and health centers for inexpensive first-line screening.

The nucleic acid to be detected may be isolated by known methods, or may be detected directly in cells, tissue samples, biological fluids (e.g., saliva, urine, blood, serum), solutions containing PCR components, solutions containing large excesses of oligonucleotides or high molecular weight DNA, and other samples, as also known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995). Methods of preparing nucleic acids for detection with hybridizing probes are well known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995).

If a nucleic acid is present in small amounts, it may be applied by methods known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995). Preferred is polymerase chain reaction (PCR) amplification.

One method according to the invention for detecting nucleic acid comprises contacting a nucleic acid with one or more types of nanoparticles having oligonucleotides attached thereto. The nucleic acid to be detected has at least two portions. The lengths of these portions and the distance(s), if any, between them are chosen so that when the oligonucleotides on the nanoparticles hybridize to the nucleic acid, a detectable change occurs. These lengths and distances can be determined empirically and will depend on the type of particle used and its size and the type of electrolyte which will be present in solutions used in the assay (as is known in the art, certain electrolytes affect the conformation of nucleic acids).

Also, when a nucleic acid is to be detected in the presence of other nucleic acids, the portions of the nucleic acid to which the oligonucleotides on the nanoparticles are to bind must be chosen so that they contain sufficient unique sequence so that detection of the nucleic acid will be specific. Guidelines for doing so are well known in the art.

Although nucleic acids may contain repeating sequences close enough to each other so that only one type of oligonucleotide-nanoparticle conjugate need be used, this will be a rare occurrence. In general, the chosen portions of the nucleic acid will have different sequences and will be contacted with nanoparticles carrying two or more different oligonucleotides, preferably attached to different nanoparticles. For example, a first oligonucleotide attached to a first nanoparticle has a sequence complementary to a first portion of the target sequence in the single-stranded DNA. A second oligonucleotide attached to a second nanoparticle has a sequence complementary to a second portion of the target sequence in the DNA. Additional portions of the DNA could be targeted with corresponding nanoparticles. Targeting several portions of a nucleic acid increases the magnitude of the detectable change.

The contacting of the nanoparticle-oligonucleotide conjugates with the nucleic acid takes place under conditions effective for hybridization of the oligonucleotides on the nanoparticles with the target sequence(s) of the nucleic acid. These hybridization conditions are well known in the art and can readily be optimized for the particular system employed. See, e.g., Sambrook et al., Molecular Cloning: A Laboratory Manual (2nd ed. 1989). Preferably stringent hybridization conditions are employed.

Faster hybridization can be obtained by freezing and thawing a solution containing the nucleic acid to be detected and the nanoparticle-oligonucleotide conjugates. The solution may be frozen in any convenient manner, such as placing it in a dry ice-alcohol bath for a sufficient time for the solution to freeze (generally about 1 minute for 100 uL of solution). The solution must be thawed at a temperature below the thermal denaturation temperature, which can conveniently be room temperature for most combinations of nanoparticle-oligonucleotide conjugates and nucleic acids. The hybridization is complete, and the detectable change may be observed, after thawing the solution.

The rate of hybridization can also be increased by warming the solution containing the nucleic acid to be detected and the nanoparticle-oligonucleotide conjugates to a temperature below the dissociation temperature (Tm) for the complex formed between the oligonucleotides on the nanoparticles and the target nucleic acid. For nanoparticle oligonuclotide conjugates, high stringency conditions may be used for hybridization since the melting transitions are extremely sharp, leading to higher specificity hybridization. Alternatively, rapid hybridization can be achieved by heating above the dissociation temperature (Tm) and allowing the solution to cool.

The rate of hybridization can also be increased by increasing the salt concentration (e.g., from 0.1 M to 0.3 M NaCl) or by using divalent salts (e.g., MgCl₂).

The detectable change that occurs upon hybridization of the oligonucleotides on the nanoparticles to the nucleic acid may be an optical change such as a color change, the formation of aggregates of the nanoparticles, or the precipitation of the aggregated nanoparticles. The optical changes can be observed with the naked eye or spectroscopically. The formation of aggregates of the nanoparticles can be observed by electron microscopy or by nephelometry. The precipitation of the aggregated nanoparticles can be observed with the naked eye or microscopically. Preferred are changes observable with an optical detection device such as a spectrophotometer or visually. Particularly preferred is a color change observable at specific wavelengths.

The observation of a color change spectrophotometrically can be performed using extremely simple instrumentation. For instance, 15 nm diameter gold probes bound to targets exhibit colorimetric shifts that are detectable in the 200-1100 nm wavelength region using a UV-visible spectrophotometer (Storhoff et. al, J. Am. Chem. Soc., 120, 1959 (1998). Any wavelength that exhibits a change in extinction upon gold probe hybridization to target may be monitored to determine the presence of the nucleic acid target. For instance, a significant change in intensity is observed at 260 and 700 nm may be monitored during target amplification for detection of PCR amplicons in real time. In addition, larger 30 nm diameter gold nanoparticles probes may be hybridized to complementary PCR amplified fragments which produces a visual colorimetric change that may be monitored spectrophotometrically (see Example 5 below) in the 200-1100 nm region. For instance, a significant change is observed in the region of 450-700 nm, and may be monitored during nucleic acid target amplification in real time. Since it is possible to monitor individual wavelengths that are responsive to gold nanoparticle probe/target hybridization and aggregation, a UV-visible spectrophotometer is not necessary. A simplified detection system that monitors a single wavelength or set of wavelengths could be used for detection and integrated with a peltier device to perform the necessary thermal cycling to form a real time PCR detection system. In addition, the gold probes may also be monitored optically via Rayleigh scattering or dynamic light scattering.

The observation of a color change with the naked eye can be made more readily against a background of a contrasting color. For instance, when gold nanoparticles are used, the observation of a color change is facilitated by spotting a sample of the hybridization solution on a solid white surface (such as silica or alumina TLC plates, filter paper, cellulose nitrate membranes, and nylon membranes, preferably a C-18 silica TLC plate) and allowing the spot to dry. Initially, the spot retains the color of the hybridization solution (which ranges from pink/red, in the absence of hybridization, to purplish-red/purple, if there has been hybridization). On drying at room temperature or 80° C. (temperature is not critical), a blue spot develops if the nanoparticle-oligonucleotide conjugates had been linked by hybridization with the target nucleic acid prior to spotting. In the absence of hybridization (e.g., because no target nucleic acid is present), the spot is pink. The blue and the pink spots are stable and do not change on subsequent cooling or heating or over time. They provide a convenient permanent record of the test. No other steps (such as a separation of hybridized and unhybridized nanoparticle-oligonucleotide conjugates) are necessary to observe the color change.

An alternate method for easily visualizing the assay results is to spot a sample of nanoparticle probes hybridized to a target nucleic acid on a cellulose acetatate membrane (e.g. 0.2 micron diameter pore size cellulose acetate membrane), while drawing the liquid through the filter. The excess, non-hybridized probes pass through the filter since they do not have an affinity for the membrane, leaving behind an observable spot comprising the aggregates generated by hybridization of the nanoparticle probes with the target nucleic acid (retained because these aggregates are larger than the pores of the filter). This technique may provide for greater sensitivity, since an excess of nanoparticle probes can be used. Unfortunately, the nanoparticle probes stick to many other solid surfaces that have been tried (silica slides, reverse-phase plates, and nylon, nitrocellulose, cellulose and other membranes), and these surfaces cannot be used.

The nanoparticle-oligonucleotide probes can be prepared by any suitable method. Suitable, but non-limiting, nanoparticles and methods are described in U.S. Pat. Nos. 4,683,195 and 4,683,202, as well as published international application nos. PCT/US01/01190, filed Jan. 12, 2001; PCT/US01/10071, filed Mar. 28, 2001; PCT/US01/46418, filed Dec. 7, 2001; and PCT/US01/25237, filed Aug. 10, 2001, which are incorporated by reference in their entirety. In the first such method, oligonucleotides are bound to charged nanoparticles to produce stable nanoparticle-oligonucleotide conjugates. Charged nanoparticles include nanoparticles made of metal, such as gold nanoparticles.

The method comprises providing oligonucleotides having bound thereto a moiety comprising a functional group which can bind to the nanoparticles. The moieties and functional groups are those described above for binding (i.e., by chemisorption or covalent bonding) oligonucleotides to nanoparticles. For instance, oligonucleotides having an alkanethiol or an alkanedisulfide covalently bound to their 5′ or 3′ ends can be used to bind the oligonucleotides to a variety of nanoparticles, including gold nanoparticles.

The oligonucleotides are contacted with the nanoparticles in aqueous solution for a time sufficient to allow at least some of the oligonucleotides to bind to the nanoparticles by means of the functional groups. Such times can be determined empirically. For instance, it has been found that a time of about 12-24 hours gives good results. Other suitable conditions for binding of the oligonucleotides can also be determined empirically. For instance, a concentration of about 10-20 nM nanoparticles and incubation at room temperature gives good results.

Next, at least one salt is added to the aqueous solution to form a salt solution. The salt can be any water-soluble salt. For instance, the salt may be sodium chloride, magnesium chloride, potassium chloride, ammonium chloride, sodium acetate, ammonium acetate, a combination of two or more of these salts, or one of these salts in phosphate buffer. Preferably, the salt is added as a concentrated solution, but it could be added as a solid. The salt can be added to the water all at one time or the salt is added gradually over time. By “gradually over time” is meant that the salt is added in at least two portions at intervals spaced apart by a period of time. Suitable time intervals can be determined empirically.

The ionic strength of the salt solution must be sufficient to overcome at least partially the electrostatic repulsion of the oligonucleotides from each other and, either the electrostatic attraction of the negatively-charged oligonucleotides for positively-charged nanoparticles, or the electrostatic repulsion of the negatively-charged oligonucleotides from negatively-charged nanoparticles. Gradually reducing the electrostatic attraction and repulsion by adding the salt gradually over time has been found to give the highest surface density of oligonucleotides on the nanoparticles. Suitable ionic strengths can be determined empirically for each salt or combination of salts. A final concentration of sodium chloride of from about 0.1 M to about 1.0 M in phosphate buffer, preferably with the concentration of sodium chloride being increased gradually over time, has been found to give good results.

After adding the salt, the oligonucleotides and nanoparticles are incubated in the salt solution for an additional period of time sufficient to allow sufficient additional oligonucleotides to bind to the nanoparticles to produce the stable nanoparticle-oligonucleotide conjugates. As will be described in detail below, an increased surface density of the oligonucleotides on the nanoparticles has been found to stabilize the conjugates. The time of this incubation can be determined empirically. A total incubation time of about 24-48, preferably 40 hours, has been found to give good results (this is the total time of incubation; as noted above, the salt concentration can be increased gradually over this total time). This second period of incubation in the salt solution is referred to herein as the “aging” step. Other suitable conditions for this “aging” step can also be determined empirically. For instance, incubation at room temperature and pH 7.0 gives good results.

The conjugates produced by use of the “aging” step have been found to be considerably more stable than those produced without the “aging” step. As noted above, this increased stability is due to the increased density of the oligonucleotides on the surfaces of the nanoparticles which is achieved by the “aging” step. The surface density achieved by the “aging” step will depend on the size and type of nanoparticles and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 10 picomoles/cm² will be adequate to provide stable nanoparticle-oligonucleotide conjugates. Preferably, the surface density is at least 15 picomoles/cm². Since the ability of the oligonucleotides of the conjugates to hybridize with nucleic acid and oligonucleotide targets can be diminished if the surface density is too great, the surface density is preferably no greater than about 35-40 picomoles/cm².

As used herein, “stable” means that, for a period of at least six months after the conjugates are made, a majority of the oligonucleotides remain attached to the nanoparticles and the oligonucleotides are able to hybridize with nucleic acid and oligonucleotide targets under standard conditions encountered in methods of detecting nucleic acid and methods of nanofabrication.

Aside from their stability, the nanoparticle-oligonucleotide conjugates made by this method exhibit other remarkable properties. See, e.g., Examples 5, 7, and 19 of published international application nos. PCT/US01/01190, filed Jan. 12, 2001; PCT/US01/10071, filed Mar. 28, 2001; PCT/US01/46418, filed Dec. 7, 2001; and PCT/US01/25237, filed Aug. 10, 2001, which are incorporated by reference in its entirety. In particular, due to the high surface density of the conjugates, they will assemble into large aggregates in the presence of a target nucleic acid or oligonucleotide. The temperature over which the aggregates form and dissociate has unexpectedly been found to be quite narrow, and this unique feature has important practical consequences. In particular, it increases the selectivity and sensitivity of the methods of detection of the present invention. A single base mismatch and as little as 20 femtomoles of target can be detected using the conjugates. Although these features were originally discovered in assays performed in solution, the advantages of the use of these conjugates have been found to extend to assays performed on substrates, including those in which only a single type of conjugate is used.

It has been found that the hybridization efficiency of nanoparticle-oligonucleotide conjugates can be increased dramatically by the use of recognition oligonucleotides which comprise a recognition portion and a spacer portion. “Recognition oligonucleotides” are oligonucleotides which comprise a sequence complementary to at least a portion of the sequence of a nucleic acid or oligonucleotide target. In this embodiment, the recognition oligonucleotides comprise a recognition portion and a spacer portion, and it is the recognition portion which hybridizes to the nucleic acid or oligonucleotide target. The spacer portion of the recognition oligonucleotide is designed so that it can bind to the nanoparticles. For instance, the spacer portion could have a moiety covalently bound to it, the moiety comprising a functional group which can bind to the nanoparticles. These are the same moieties and functional groups as described above. As a result of the binding of the spacer portion of the recognition oligonucleotide to the nanoparticles, the recognition portion is spaced away from the surface of the nanoparticles and is more accessible for hybridization with its target. The length and sequence of the spacer portion providing good spacing of the recognition portion away from the nanoparticles can be determined empirically. It has been found that a spacer portion comprising at least about 10 nucleotides, preferably 10-30 nucleotides, gives good results. The spacer portion may have any sequence which does not interfere with the ability of the recognition oligonucleotides to become bound to the nanoparticles or to a nucleic acid or oligonucleotide target. For instance, the spacer portions should not sequences complementary to each other, to that of the recognition olignucleotides, or to that of the nucleic acid or oligonucleotide target of the recognition oligonucleotides. Preferably, the bases of the nucleotides of the spacer portion are all adenines, all thymines, all cytidines, or all guanines, unless this would cause one of the problems just mentioned. More preferably, the bases are all adenines or all thymines. Most preferably the bases are all thymines.

It has further been found that the use of diluent oligonucleotides in addition to recognition oligonucleotides provides a means of tailoring the conjugates to give a desired level of hybridization. The diluent and recognition oligonucleotides have been found to attach to the nanoparticles in about the same proportion as their ratio in the solution contacted with the nanoparticles to prepare the conjugates. Thus, the ratio of the diluent to recognition oligonucleotides bound to the nanoparticles can be controlled so that the conjugates will participate in a desired number of hybridization events. The diluent oligonucleotides may have any sequence which does not interfere with the ability of the recognition oligonucleotides to be bound to the nanoparticles or to bind to a nucleic acid or oligonucleotide target. For instance, the diluent oligonulceotides should not have a sequence complementary to that of the recognition olignucleotides or to that of the nucleic acid or oligonucleotide target of the recognition oligonucleotides. The diluent oligonucleotides are also preferably of a length shorter than that of the recognition oligonucleotides so that the recognition oligonucleotides can bind to their nucleic acid or oligonucleotide targets. If the recognition oligonucleotides comprise spacer portions, the diluent oligonulceotides are, most preferably, about the same length as the spacer portions. In this manner, the diluent oligonucleotides do not interefere with the ability of the recognition portions of the recognition oligonucleotides to hybridize with nucleic acid or oligonucleotide targets. Even more preferably, the diluent oligonucleotides have the same sequence as the sequence of the spacer portions of the recognition oligonucleotides.

As can be readily appreciated, highly desirable nanoparticle-oligonucleotide conjugates can be prepared by employing all of the methods described above. By doing so, stable conjugates with tailored hybridization abilities can be produced.

In the present invention, the nanoparticle probes are used to monitor the PCR amplification system. Prior to introduction into the PCR reaction, the nanoparticle probes are preferably contacted with a protective agent. Contacting nanoparticle probes with a protective agent is desirable to prevent or substantially reduce inactivation of nucleic acid amplification components (such as taq polymerase enzymes in PCR) so as to avoid or substantially avoid any interference to the amplification reaction by the addition of the nanoparticle probes. Without being bound by any theory of operation, it is believed that amplification enzymes such as PCR enzyme taq polymerase may bind covalently or non-covalently to an unmodified gold surface. By contacting the nanoparticle surfaces areas not having oligonucleotides bound thereto with a protective agent, it is believed that such surfaces may be passivated. Any suitable concentration of protective agent may be used that would not interfere with the nucleic acid amplification reaction and that would allow for passivation of a sufficient portion of any unlabeled nanoparticle surfaces so as to prevent any interference by the nanoparticles with the amplification reaction. The protective agent in aqueous solution is admixed with the aqueous nanoparticle probe mixture at room temperature just prior to use. The concentration of protective agent generally ranges from about 0.001% to about 2% (w/v), usually about 0.001% to about 0.05% (w/v), of passivating agent in the nanoparticle probe mixture. Suitable, but non-limiting, passivating agents include albumin such as bovine serum albumin (BSA), casein, streptavidin, polyethylene glycol (PEG), acid terminated and amine terminated thiols (such as mercaptourdecanoic acid and mercaptoethylamine), gelatin such as fish gelatin, organic molecules having one or more thiol groups, DNA such as salmon sperm DNA, detergents such as sodium docecyl sulfate or Tween 20, other proteins and small thiol containing peptides such as glutathione. In practicing this invention, BSA is preferred because it is inexpensive, commercially available in purified form, and robust.

In another embodiment of the invention, kits for detecting amplified nucleic acid targets and for performing real time nucleic acid amplification monitoring are provided. The kits include nanoparticle-oligonucleotide conjugates and may also contain other reagents and items useful for detecting nucleic acid. The reagents may include nucleic acid amplification, e.g., PCR, reagents, hybridization reagents, buffers, etc. Other items which may be provided as part of the kit include a solid surface (for visualizing hybridization) such as a TLC silica plate, microporous materials, syringes, pipettes, cuvettes, containers, and a thermocycler (for controlling hybridization and de-hybridization temperatures). Reagents for functionalizing the nucleotides or nanoparticles may also be included in the kit.

C. Method of the Present Invention

The general method of the invention involves an all-in-one assay for detecting a target polynucleotide in a sample during amplification of the polynucleotide, preferably by the polymerase chain reaction (PCR). Detection is accomplished by monitoring amplification of the target DNA using a nanoparticle system, particularly a nanoparticle detection system that employs nanoparticle probes that have been contacted with a protective agent. Typically, the method commences with at least one cycle of amplification of the target polynucleotide. After at least one cycle of amplification, the nanoparticle probes are allowed to bind to the target polynucleotide and a signal measurement is taken. Additional luminescence measurements are taken after subsequent cycles. These measurements are then analyzed and used to determine the presence of the target polynucleotide.

Amplification of the target polynucleotide is carried out by an amplification method. A preferred amplification method is the polymerase chain reaction. Mullis, U.S. Pat. No. 4,683,202 (1987). However, other amplification methods are known, including the ligase chain reaction. EP-A-320 308; U.S. Pat. No. 5,427,930. The requirements of the nucleic acid amplification method are that it is capable of amplifying the target polynucleotide many times and the method can be paused so that the amplified product can be detected during the amplification process. Finally, the amplification method cannot destroy the detection system during rounds of amplification.

The nucleic acid amplification method typically occurs through a repetitive series of cycles, preferably temperature cycles. The first step in the amplification process is typically separation of the two strands of the polynucleotide so that they can be used as templates, unless the target polynucleotide is single-stranded wherein separation is not necessary. Another exception to the usual first step separation occurs when the target polynucleotide is RNA instead of DNA. In this situation a reverse transcriptase is typically used to synthesize a DNA strand from the RNA template before the strand separation step. The strand separation can be accomplished by any suitable method including physical, chemical or enzymatic means. One preferred physical method of separating the strands of the nucleic acid involves heating the nucleic acid until it is denatured. Typical heat denaturation may involve temperatures ranging from about 80 C. to 105 C. for times ranging from about 1 to 10 minutes. Other methods of strand separation are known in the art including separation using enzymes known as helicases. Cold Spring Harbor Symposia on Quantitative Biology, Vol. XLIII “DNA: Replication and Recombination” (New York: Cold Spring Harbor Laboratory, 1978), B. Kuhn et al., “DNA Helicases”, pp. 63-67; C; Radding, Ann. Rev. Genetics, 16: 405-37 (1982).

When the complementary strands of the target polynucleotide are separated, whether the nucleic acid was originally double or single stranded, the strands are ready to be used as a template for the synthesis of additional polynucleotide strands. This synthesis can be performed using any suitable method. Generally it occurs in a buffered aqueous solution, preferably at a pH of 7-9, most preferably about 8. Preferably, a molar excess of two oligonucleotide primers, a forward primer and a reverse primer, is added to the buffer containing the separated template strands. It is understood, however, that the amount of complementary strand may not be known, for example if the process herein is used for target polynucleotides of unknown concentrations in patient samples. As a practical matter, however, the amount of primer added will generally be in molar excess over the amount of complementary strand (template). The deoxyribonucleoside triphosphates dATP, dCTP, dGTP and dTTP and an agent for inducing or catalyzing the primer extension are also added to the synthesis mixture in adequate amounts and the resulting solution is heated to about 90 C.-100 C. for from about 1 second to 5 minutes, preferably from 10 to 30 seconds, most preferably 15 seconds. The agent for inducing or catalyzing the primer extension reaction is typically a thermostable DNA polymerase of which many are known in the art. Preferably the thermostable polymerase is Taq polymerase, most preferably it is Pfu, the DNA polymerase from Pyrococcus furiosis, which has an exceptionally low error rate. After this heating period the solution is allowed to cool to a temperature which allows primer hybridization. The temperature is then typically changed to a temperature that will allow the polymerase-catalyzed primer extension reaction to occur under conditions known in the art. This synthesis reaction may occur at from room temperature up to a temperature above which the inducing agent no longer functions efficiently. The temperature is typically higher than that used for annealing the forward and reverse primers to the template. One of ordinary skill in the art can readily use empirical means to determine the appropriate denaturation and annealing temperatures for any particular amplification reaction mixture and program a thermocycler accordingly. Generally, the synthesis will be initiated at the 3′ end of each primer and proceed in the 5′ direction along the template strand until synthesis terminates.

The newly synthesized strand and its complementary nucleic acid strand form a double-stranded molecule which is used in succeeding rounds of synthesis by repeating the strand-separation, primer annealing, and extension steps described above. These steps can be repeated as often as needed. The amount of the specific nucleic acid sequence produced will accumulate in an exponential fashion. Therefore, the amplification process includes an exponential phase that typically ends when one or more of the reactants are exhausted.

In one preferred embodiment, a “hot start” method is used to improve specificity. In general, in this preferred but non-essential embodiment at least one component that is essential for polymerization is not present until the reaction is heated to the annealing or extension temperatures. This method, termed “hot start,” improves specificity and minimizes the amplification of unspecific DNA. The hot start method also minimizes the formation of “primer-dimers,” which are double-stranded PCR products resulting from extension of one primer using the other primer as template. In one embodiment of the hot start method, DNA polymerase can be added to the PCR reaction mixture after both the primer and template are added and the temperature has been increased appropriately. Alternatively, for example, after the temperature has been increased appropriately, the enzyme and primer are added last or the PCR buffer or template plus buffer are added last. Finally, a commercially available wax beads such as PCR Gems® (PE biosystems, Foster City, Calif., USA) may be used in a hot start method. The wax beads melt and form a barrier at the top of the PCR reaction mixture. The enzyme is added to the top of the wax barrier, and thermal cycling is continued wherein the wax melts again and allows mixing of the polymerase with the rest of the mixture and hot start amplification begins.

In other embodiment of the hot start method, thermal stable DNA polymerases which activate upon heating to high temperatures (e.g., above 60° C.) may be used. Suitable thermal stable DNA polymerases include the ones described in Roche U.S. Pat. No. 5,677,152. Alternatively, a hot start method could utilize an antibody against the thermal stable DNA polymerase which inactivates the polymerase until the antibody comes off the polymerase at relatively high temperatures. See for instance, Kodak U.S. Pat. No. 5,338,671.

In the present invention, a nanoparticle detection system is utilized to monitor the PCR reaction. The nanoparticle detection system components are added to the amplification reaction mixture before or during the amplification process. The presence of the nanoparticle detection system must not destroy or interfere with the amplification process. Signal analysis can be carried out at a variety of temperatures, typically the chemiluminescence analysis is performed at temperatures between 20° C. and 75° C., preferably 37° C. The desired temperature range will depend on the length of the probe, bead oligo base pairing, and probe/target base pairing.

Signal measurement after a certain number of cycles are translated into a qualitative determination of the presence of the target polynucleotide or a quantitative determination of the amount of target polynucleotide present in the sample. In one embodiment, qualitative determinations are made by comparing the signal produced emitted after various amplification cycles for the sample compared with a control without target polynucleotide. Typically, quantitative determinations involve the generation of a standard curve using measurements taken from samples with known amounts of target polynucleotide. In a preferred embodiment the amount of target polynucleotide in a sample is generated by determining a threshold cycle number at which the signal generated from amplification of the target polynucleotide in a sample reaches a fixed threshold value above a baseline value. This cycle number is compared to a standard curve of threshold cycle numbers determined using target polynucleotides of various known concentrations to yield the quantity of target polynucleotide in the sample. Various data reduction techniques including point to point and curve fitting techniques known in the art can be used for this analysis.

The method of the present invention is useful in many of the situations in which PCR is useful, including the analysis of a patient's own genome. In a preferred embodiment of the present invention various infectious diseases, for humans and animals, can be diagnosed by the presence in clinical samples of specific target polynucleotides characteristic of the causative microorganism. These microorganisms include, but are not limited to, bacteria, such as Salmonella, Chlamydia, and Neisseria; viruses, such as the hepatitis viruses and Human Immunodeficiency Virus; and protozoan parasites, such as the Plasmodium responsible for malaria. The invention is especially effective in detecting disease-causing microorganisms because it can detect very small numbers of target polynucleotides of the pathogenic organism.

### EXAMPLES

The invention is demonstrated further by the following illustrative examples. The examples are offered by way of illustration and are not intended to limit the invention in any manner. In these examples all percentages are by weight if for solids and by volume if for liquids, and all temperatures are in degrees Celsius unless otherwise noted.

### Example 1

Preparation of Nanoparticle-Oligonucleotide Conjugate Probes

In this Example, a representative nanoparticle-oligonucleotide conjugate detection probe was prepared for the use in the PCR amplification of a MTHFR target.

(a) Preparation of Gold Nanoparticles

Gold colloids (13 nm diameter) were prepared by reduction of HAuCl₄ with citrate as described in Frens, 1973, Nature Phys. Sci., 241:20-22 and Grabar, 1995, Anal. Chem.67:735. Briefly, all glassware was cleaned in aqua regia (3 parts HCl, 1 part HNO₃), rinsed with Nanopure H₂O, then oven dried prior to use. HAuCl₄ and sodium citrate were purchased from Aldrich Chemical Company. Aqueous HAuCl₄ (1 mM, 500 mL) was brought to reflux while stirring. Then, 38.8 mM sodium citrate (50 mL) was added quickly. The solution color changed from pale yellow to burgundy, and refluxing was continued for 15 min. After cooling to room temperature, the red solution was filtered through a Micron Separations Inc. 1 micron filter. Au colloids were characterized by UV-vis spectroscopy using a Hewlett Packard 8452A diode array spectrophotometer and by Transmission Electron Microscopy (TEM) using a Hitachi 8100 transmission electron microscope. Gold particles with diameters of 13-17 nm will produce a visible color change when aggregated with target and probe oligonucleotide sequences in the 10-80 nucleotide range.

(b) Synthesis of Steroid Disulfide Modified Oligonucleotides (SDO)

Oligonucleotides complementary to segments of the APC gene DNA sequence were synthesized on a 1 micromole scale using a Milligene Expedite DNA synthesizer in single column mode using phosphoramidite chemistry. Eckstein, F. (ed.) Oligonucleotides and Analogues: A Practical Approach (IRL Press, Oxford, 1991). All solutions were purchased from Milligene (DNA synthesis grade). Average coupling efficiency varied from 98 to 99.8%, and the final dimethoxytrityl (DMT) protecting group was removed from the oligonucleotides so that the steroid disulfide phosphoramidite could be coupled.

To facilitate hybridization of the probe sequence with the target, a deoxyadenosine oligonucleotide (da₂₀) was included on the 5′ end in the probe sequence as a spacer.

To generate 5′-terminal steroid-cyclic disulfide oligonucleotide derivatives (see Letsinger et al., 2000, Bioconjugate Chem. 11:289-291 and PCT/US01/01190 (Nanosphere, Inc.), the disclosure of which is incorporated by reference in its entirety), the final coupling reaction was carried out with a cyclic dithiane linked epiandrosterone phosphoramidite on Applied Biosystems automated Expedite 8909 synthesizer, a reagent that prepared using 1,2-dithiane-4,5-diol, epiandrosterone and p-toluenesulphonic acid (PTSA) in presence of toluene. The phosphoramidite reagent may be prepared as follows: a solution of epiandrosterone (0.5 g), 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the reside taken up in ethyl acetate. This solution was washed with water, dried over sodium sulfate, and concentrated to a syrupy reside, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. Recrystallization from pentane/ether afforded a white powder, mp 110-112° C.; ¹H NMR, δ3.6 (1H, C³OH), 3.54-3.39 (2H, m 2OCH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in the minimum amount of dichloromethane, precipitated at −70° C. by addition of hexane, and dried under vacuum; yield 100 mg; ³¹P NMR 146.02. The epiandrosterone-disulfide linked oligonucleotides were synthesized on Applied Biosystems automated gene synthesizer without final DMT removal. After completion, epiandrosterone-disulfide linked oligonucleotides were deprotected from the support under aqueous ammonia conditions and purified on HPLC using reverse phase column.

Reverse phase HPLC was performed with a Dionex DX500 system equipped with a Hewlett Packard ODS hypersil column (4.6×200 mm, 5 mm particle size) using 0.03 M Et₃NH⁺ OAc⁻ buffer (TEAA), pH 7, with a 1%/min. gradient of 95% CH₃CN/5% TEAA. The flow rate was 1 mL/min. with UV detection at 260 nm. Preparative HPLC was used to purify the DMT-protected unmodified oligonucleotides. After collection and evaporation of the buffer, the DMT was cleaved from the oligonucleotides by treatment with 80% acetic acid for 30 min at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by reverse phase HPLC.

(c) Attachment of SDOs to Gold Nanoparticles

A solution of ˜13.75 nM gold nanoparticles (˜15 nm diameter) was prepared using the citrate reduction method.¹ The gold nanoparticle probes were prepared by loading the ˜15 nm diameter gold particles (˜13.75 nM) with steroid disulfide modified oligonucleotides using a modification of previously developed procedures.³ Briefly, 4 nmol of SDO was added per 1 mL of 13.7 nM gold nanoparticle buffered at 10 mM phosphate (pH 7) and incubated for 15 hours at room temperature. The solution was raised to 0.3 M NaCl, 10 mM phosphate (pH 7) using 4 M NaCl, 10 mM phosphate (pH 7) and incubated for 8 hours. The solution was then raised to 0.8 M NaCl, 10 mM phosphate (pH 7) using the same buffer and incubated for 42 hours. The SDO-gold nanoparticle conjugates were isolated with a Beckman Coulter Microfuge 18 by centrifugation at 14000 rpm for 25 minutes. After centrifugation, a dark red gelatinous residue remained at the bottom of the eppendorf tube. The supernatant was removed, and the conjugates were redispersed in 0.1 M NaCl, 10 mM phosphate (pH 7) (original colloid volume) and recentrifuged, followed by redispersion in 0.1 M NaCl, 10 mM phosphate (pH 7) at a final nanoparticle concentration of 10 nM. For the PCR experiments, the gold conjugates were recentrifuged at 14000 rpm for 25 minutes, washed with water as described above, and redispersed in 25 mM Tris.HCl buffer (pH 8) at a final nanoparticle concentration of 10 nM. SDO gold conjugates with BSA were prepared by mixing 20 uL of a 10× BSA solution (5 mg/mL) with 200 uL of the SDO modified gold probe at room temperature, which was then used directly in the PCR amplification experiments.

The following nanoparticle-oligonucleotide conjugates specific for segments of the APC gene of the human genome were prepared in this manner:

Probe APC 1-WT: gold-S′-5′-[a₂₀-gcagaaataaaag-3′]ₙ (SEQ ID NO: 1)

Probe APC 1-MUT: gold-S′-5′-[a₂₀-gcagaaaaaaaag-3′]ₙ (SEQ ID NO:2)

Probe APC 2: gold-S′-5′-[a₂₀-aaaagattggaacta-3′]ₙ (SEQ ID NO:3)

S′ indicates a connecting unit prepared via an epiandrosterone disulfide group; n indicates that a number of oligonucleotides are attached to each gold nanoparticle.

### Example 2

Determination of Melting Profiles of Nanoparticle-Oligonucleotide Detection Probes

In this Example, a melting profile study was initially performed with APC gene nanoparticle probe sequences prepared as described in Example 1 to demonstrate that the probes hybridize to complementary targets in a highly specific manner due to sharp melting transitions, and that the transitions may be monitored by UV-visible spectrophotometry. The target sequences used for the melting profile study are shown below in Table 1.

The synthetic target sequences were prepared using standard phosphoramidite chemistry (Eckstein, F. (ed.) Oligonucleotides and Analogues: A Practical Approach (IRL Press, Oxford, 1991), and have the same sequence as a 78 bp PCR amplicon from the APC gene used for testing (vide infra). For the homogeneous melting assay, a two probe system was used, where the recognition sequence on the first probe (APC1-X, where X=MUT or WT) contains a single base mutation site and therefore serves as the differentiating element, and the recognition sequence on the second probe (APC 2) is located downstream from the first probe. Probe 1 is designed to have a lower melting temperature than probe 2 so that probe 1 will dissociate from the target at a lower temperature. Within the table, the primer binding regions are shown in bold, and the probe binding regions are underlined. The single base mutation location is highlighted.

TABLE 1

Sequences of synthetic targets and PCR amplicons probes used for assay
development.

| | | |
|---|---|---|
| MTHFR gene | 5′TATTGGCAGGTTACCCCAAAGGCCACCCCGAAGCAGGGAGCTTTGAGGCTGACCTG | [SEQ ID NO. 4] |
| 119 PCR | AAGCACTTGAAGGAGAAGGTGTCTGCGGGAGCCGATTTCATCATCACGCAGCTTTTCT | |
| amplicon | TTGAG 3′ | |
| APC gene 78 | 5′CGC TCA CAG GAT CTT CAG CTG ACC TAG TTC CAA TCT TTT CTT | (SEQ ID NO:5) |
| base sequence | TTA TTT CTG CTA TTT GCA GGG TAT TAG CAG AAT CTG 3′ | |
| −Wild type | | |
| (1) | | |
| APC gene 78 | 5′CGC TCA CAG GAT CTT CAG CTG ACC TAG TTC CAA TCT TTT CTT | |
| base sequence | TTT TTT CTG CTA TTT GCA GGG TAT TAG CAG AAT CTG 3′ | (SEQ ID NO: 6) |
| Mutant (2) | | |

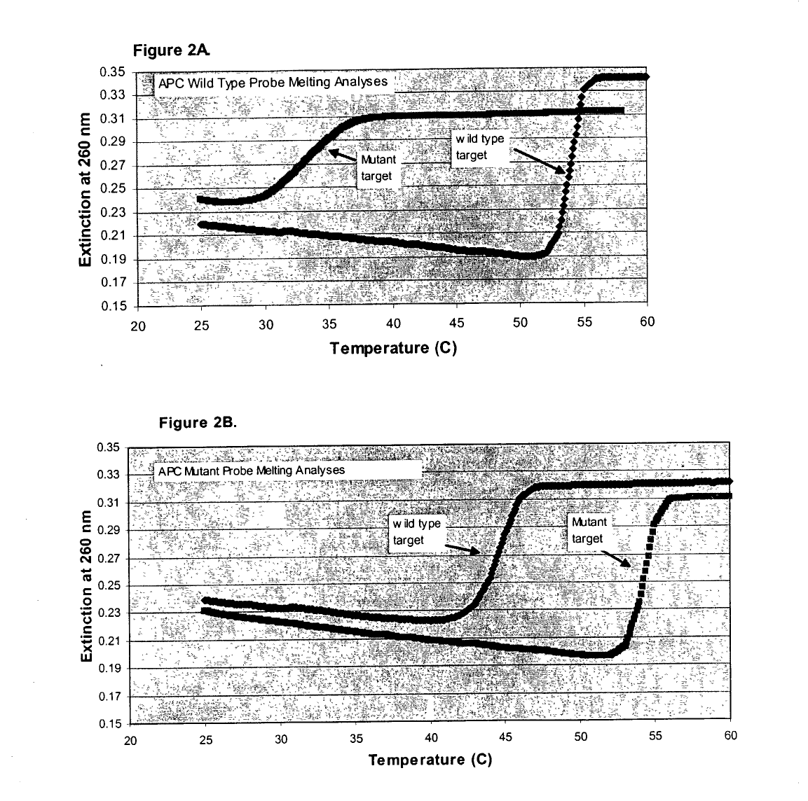

Initially, melting analyses were performed with both the wild type (SEQ ID NO: 1 and 3) and mutant (SEQ ID NO: 2 and 3) probe sets using the 78 base single stranded synthetic targets (SEQ ID NO: 5 and 6) as shown in FIG. 2. For each probe set, the perfectly complementary target and the single base mismatch target were examined to determine if the probe sets could differentiate the appropriate targets on the basis of thermal denaturation as observed in previous systems. For this test, a solution containing 600 pM of each probe was mixed with 12 nM of the appropriate target in 5×SSC (target: probe ratio of 20:1) and frozen in a dry ice bath to accelerate hybridization, followed by thermal denaturation analysis. As observed in previous studies, the probe/target complexes exihibited sharp melting transitions that occurred over a few degrees. Therefore, the perfectly matched and single base mismatched targets were easily distinguishable via thermal denaturation analysis using nanoparticle probes. For the wild type probe set (SEQ ID NO: 1 and 3), the difference in the thermal denaturation temperatures (Tm's) of the wild type (SEQ ID NO: 5) (Tₘ=54.0° C.) and mutant targets (SEQ ID NO: 6) (Tₘ=35.9° C.) was ˜18° C., which is extremely large for a single base mismatch. Using the mutant probe set (SEQ ID NO: 2 and 3), a substantial difference in Tₘ(Tₘ=˜9° C.) was observed when comparing the melting analyses from the solutions containing the mutant (SEQ ID NO: 6) (Tₘ=54.3° C.) and wild type (SEQ ID NO: 5) (Tₘ=44.8° C.) targets. This initial melting analysis data indicates that the probe sequences are highly specific and capable of differentiating single base mismatched target sequences over a range of temperatures due to the sharp melting transitions. In addition, it indicates that optical changes associated with gold probe/target hybridization may be used to monitor the presence of specific DNA sequences through melting profile analysis as is performed with fluorophore in real time PCR analysis. The advantage of the gold nanoparticle probe system is that the sharp melting transitions will enable single base discrimination superior to fluorescence technology.

### Example 3

PCR Amplification in the Presence of Nanoparticle Probes

In this Example, PCR amplification reactions of the MTHFR gene segment (SEQ ID NO: 4) were carried out in the presence of unpassivated and passivated non-complementary nanoparticle probes (SEQ ID NO: 1 and 3) to illustrate the effect of the nanoparticle probes (in passivated and unpassivated forms) on the PCR amplification reaction. The APC nanoparticle-oligonucleotide probes used in these experiments were prepared as described in Example 1.

(a) PCR Experimental Procedure

The PCR amplification was carried out with 25 μl reaction mixtures containing 100 ng of human genomic DNA, 1× PCR Buffer II (Perkin Elmer), 1.5 mM MgCl₂, 2 mM each deoxynucleoside triphosphate (dATP, dGTP, dCTP, and dUTP), 0.16 μM each oligonucleotide primer with 1 unit of AmpliTaqGold® polymerase (Perkin Elmer), and the specified amount of gold conjugate (with or without BSA). Thermal cycling was performed with a GeneAmp PCR System 2400® (Perkin Elmer). Following enzyme activation at 95° C. for 10 min, PCR was performed for 35 cycles, each cycle consisting of 94° C. for 30 s, annealing at 55° C. for 30 s, extension at 72° C. for 60 s, and final extension at 72° C. for 10 min. The 119 bp PCR amplicon was separated on a 2.0% Amplisize®/Agarose gel (BIORAD), stained with ethidium bromide, and visualized under UV light.

The spot test assay was performed in a 15 microliter volume in 1× PCR buffer at 2.5 mM MgCl₂ with 600 pM of each gold conjugate. The solution was heated to 95° C. for four minutes, frozen in a dry ice bath for three minutes, thawed on ice for ten minutes, and a five microliter aliquot was spotted onto a nylon membrane under vacuum with a glass micropipette, and the color visualized by eye for detection.

(b) Results

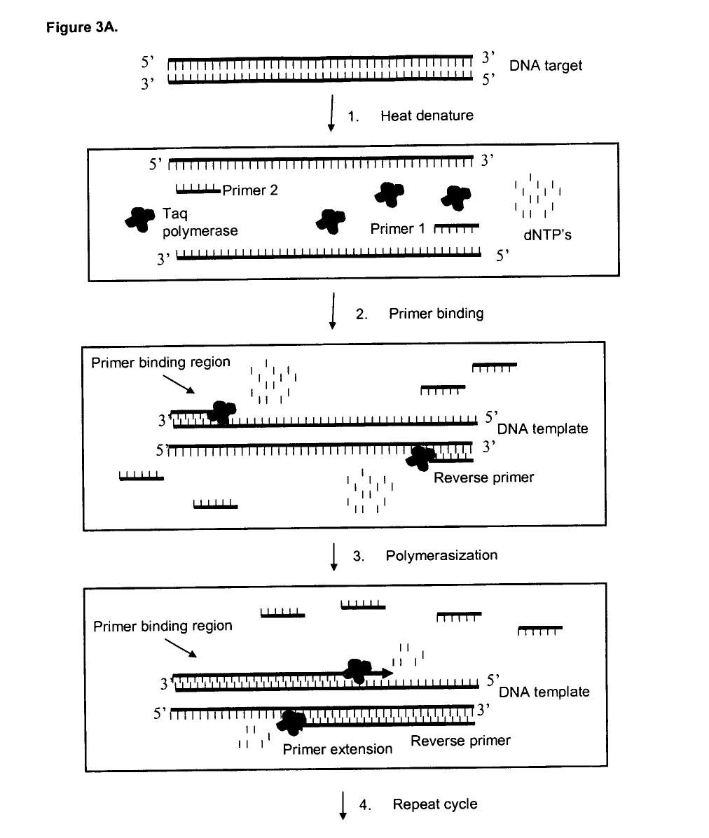

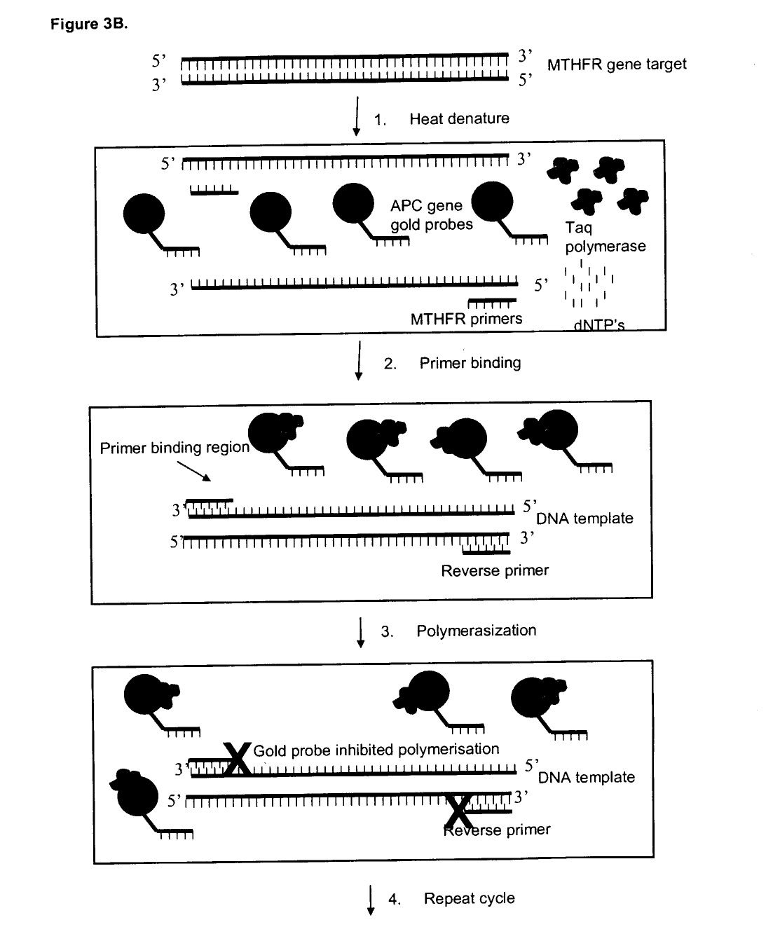

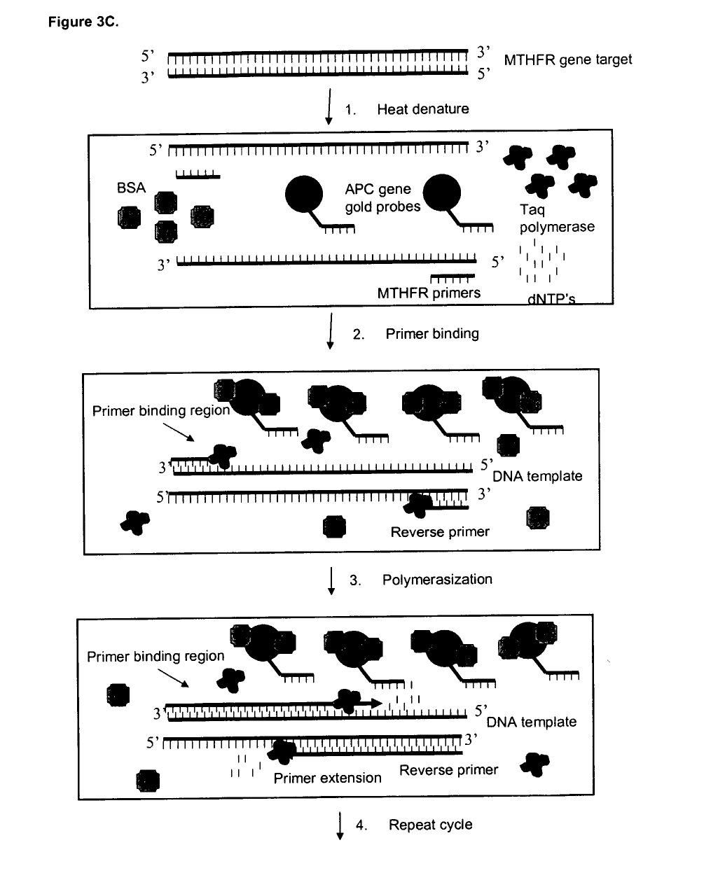

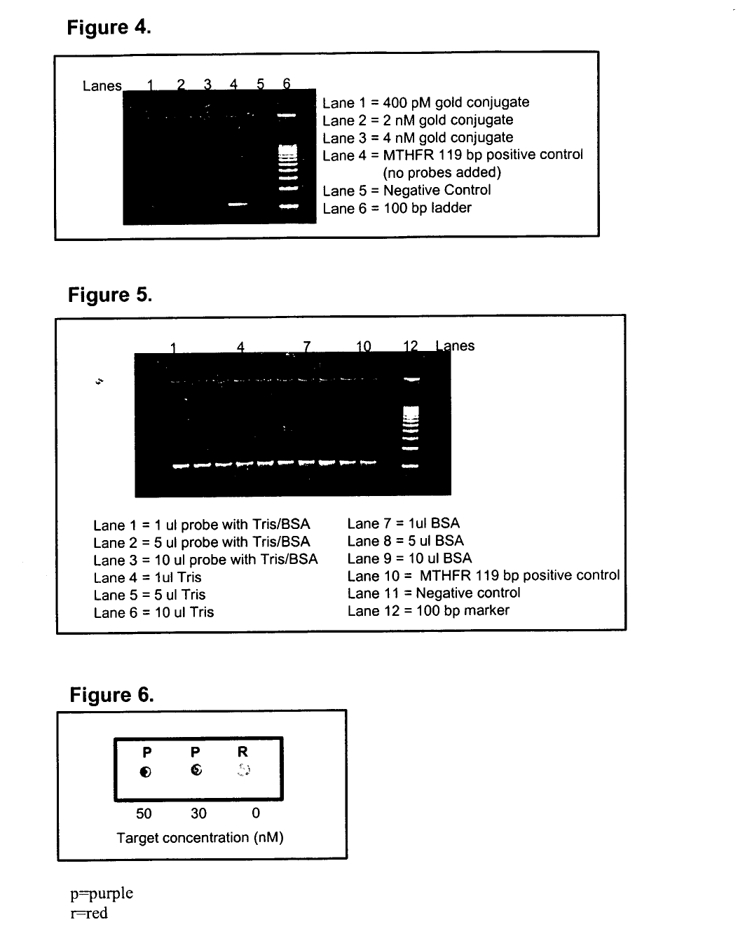

A 1:1 mixture of APC gene gold probe sequences 1-WT and 2 (SEQ ID NO: 1 and 3) in Tris buffer (pH 8) was placed directly into a PCR reaction for the MTHFR gene (119 base pair PCR amplicon) (SEQ ID NO: 4)) at final gold probe concentrations of 400 pM, 2 nM, and 4 nM, FIGS. 3B and 4. A standard PCR reaction for the MTHFR gene performed without gold conjugate served as a positive control as shown in FIG. 3A, while a PCR reaction with no template DNA served as a negative control. The positive control solution displayed an intense band on a gel stained with ethidium bromide corresponding to 119 base pairs when compared to a 100 base pair ladder, FIG. 4. By comparison, the same PCR reaction containing 400 pM of gold conjugate displayed a much fainter band at the same position, and at higher gold conjugate concentrations, no gel bands were visible, FIG. 4. These results clearly indicate that the gold conjugates inhibit the PCR reaction under standard PCR conditions as shown in FIG. 3B.

The MTHFR gene PCR reaction was performed with the same gold conjugates (1 and 2) dispersed in Tris buffer with added BSA (500 ug/mL) at final probe concentrations of 360 pM, 1.8 nM, and 3.6 nM (final BSA concentration reaction scales according to amount of probe added as illustrated in FIG. 3C. A positive control PCR reaction under standard PCR conditions was performed along with control solutions containing added BSA or Tris buffer without the gold probes. As shown in FIG. 5, the solutions containing the different concentrations of gold conjugates with BSA exhibit a gel band intensity that is similar to the positive control. This indicates that the BSA enables the PCR amplification reaction to take place in the presence of the gold probes without inhibition as shown in FIG. 3C. Therefore, the presence of non-specific proteins such as BSA enables Taq polymerase to function with the added gold nanoparticle probes. The non-specific proteins presumably bind to the gold nanoparticle surface during the PCR reaction further passivating the gold nanoparticle and ultimately preventing Taq polymerase from binding to the gold nanoparticle probes, which would inhibit the PCR amplification process.

A spot test assay was performed with gold conjugates 1-WT and 2 (SEQ ID NO: 1 and 3) dispersed in Tris/BSA (500 ug/mL) and the complementary APC gene target sequence (SEQ ID NO: 5 in Table 1) to demonstrate probe functionality in the presence of BSA, FIG. 6. A purple color was observed for the solutions containing the APC gene target sequence 1 (30 or 50 nM) when spotted onto a nylon membrane, while a red color was observed for the negative control solution which contained all reaction components except the target. The purple color indicates gold probe hybridization to the target, which demonstrates that the gold probe retain their hybridization and aggregation properties in the presence of BSA.

### Example 4

Detection of PCR-Amplified APC Gene Sequences with Gold Nanoparticle Probes

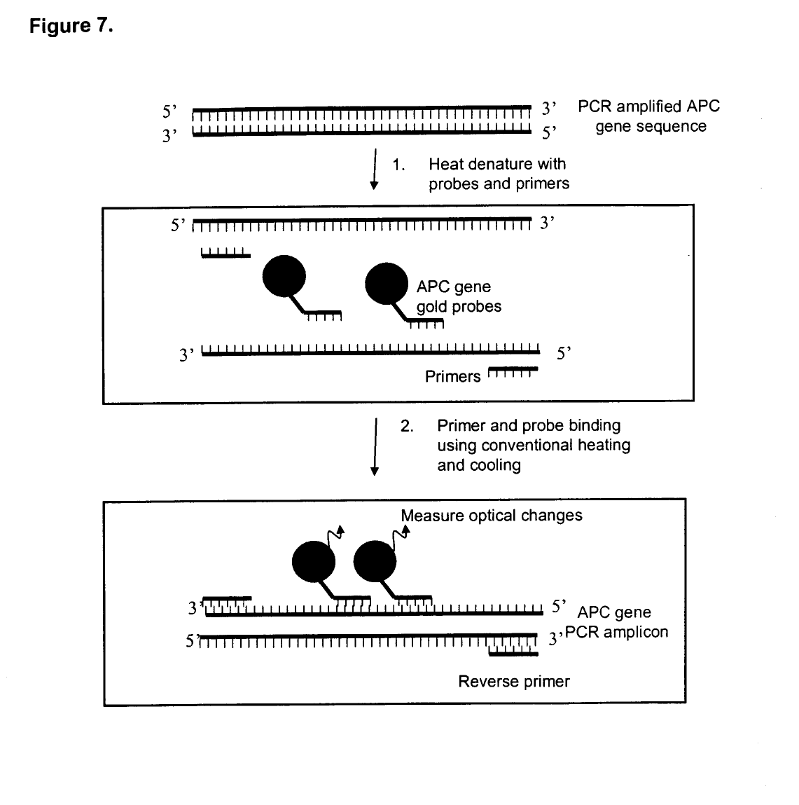

In order to achieve integrated nanoparticle probe hybridization and nucleic acid amplification as described for real time detection, it was first necessary to demonstrate that the binding of gold nanoparticle probes to a PCR amplicon could be monitored via an optical readout as shown in FIG. 7. In this Example, PCR amplified fragments of the APC gene segment were detected in solution with gold nanoparticle probes using a spectrophotometer to demonstrate the utility of nanoparticle probes in monitoring PCR reactions. The APC nanoparticle-oligonucleotide probes were prepared as described below in the experimental procedure.

(a) Experimental Procedure:

PCR amplification of the 78 base pair APC gene sequence shown in Table 1 above was carried out with 50 μl reaction mixtures containing 2 ul of 1 pM 78 base APC gene target (SEQ ID NO: 5 and6), 1× PCR Buffer II (Perkin Elmer), 1.5 mM MgCl₂, 2 mM each deoxynucleoside triphosphate (dATP, dGTP, dCTP, and dUTP), 0.16 μM each oligonucleotide primer with 1 unit of AmpliTaqGold® polymerase (Perkin Elmer). Thermal cycling was performed with a GeneAmp PCR System 2400® (Perkin Elmer). Following enzyme activation at 95° C. for 10 min, PCR was performed for 35 cycles, each cycle consisting of 94° C. for 30 s, annealing at 55° C. for 30 s, extension at 72° C. for 60 s, and final extension at 72° C. for 10 min. The 78 bp PCR amplicon was separated on a 2.0% Amplisize®/Agarose gel (BIORAD), stained with ethidium bromide, and visualized under UV light. A GFX™ PCR purification kit (Amersham Pharmacia Biotech) was used to remove salts, enzyme, unincorporated nucleotides and primers. In addition, the yield of the 78 base APC gene PCR amplicons was measured using the EZ Load Precision Molecular Mass Standard (BIORAD). Based on band intensity and molecular weight of the PCR product, it was estimated that the 78 base amplicon yielded approximately 20 ng of DNA or ˜150 nM.

30 nm diameter gold nanoparticles were purchased through Vector Labs (nanoparticles are prepared by British Biocell International). Steroid disulfide modified oligonucleotides of the APC gene sequences (1-WT, 1-MUT, and 2) in Table 1 were synthesized as described in Example 1. The SDO modified 30 nm diameter gold nanoparticle probes were prepared by adding 8 nmol of SDO to 10 mL of the 30 nm diameter gold nanoparticle. After incubation for >12 hours, 4M NaCl, 10 mM phosphate (pH 7) (4 M PBS) and 0.1 M sodium phosphate buffer (pH 7) was added to the solution to a final concentration of 0.1 M NaCl, 10 mM phosphate (pH 7), and incubated an additional 16-24 hours. Additional 4 M PBS was then added to bring the solution to 0.3 M NaCl, 10 mM phosphate (pH 7) and incubated for an additional 24-40 hours. The 30 nm diameter gold probes were isolated by centrifugation at 5000 rpm (2200 rcf) for 20 minutes, washed with 8 mL of 50 mM Tris (pH 8), and redispersed in 800 ul of 50 mM Tris (pH 8). After isolation, the concentration of the probe solution was adjusted to 2 nM.

UV-visible spectroscopy was performed using an Agilent 8453 series spectrophotometer equipped with a peltier temperature controller. Five microliters of the gold probe samples were diluted to 150 microlites with hybridization buffer and the UV-visible spectrum was recorded.

(b) Results and Discussion

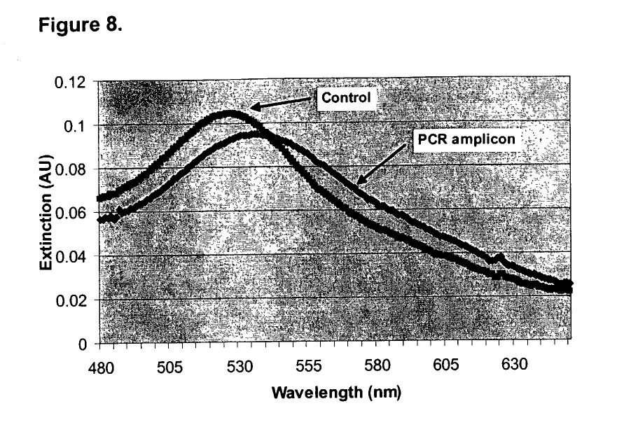

The 30 nm diameter gold probes loaded with APC-1 WT and APC 2 (SEQ ID NO: 1 and 3, respectively) were initially used for the detection of the 78 base pair wild type PCR amplicons (SEQ ID NO: 5). In the experiment, the PCR amplicon (˜37.5 nM) was mixed with the 30 nm gold APC gene probes (1-WT and 2, final concentration of 500 pM for each probe) at 0.375 M NaCl, 3.1 mM MgCl₂, 0.002% Tween 20 with 1 uM of each APC gene primer. A negative control solution containing all reaction components except for target was utilized for comparison. The solution was heated to 95° C. for five minutes followed by hybridization at 25° C. for an additional five minutes, which resulted in a solution color change from red to purple that was detectable with the naked eye. By comparison, a control solution containing no target retained its red solution color. A UV-visible spectrum of the control and target solutions was recorded to detect the optical changes, FIG. 8. A red shift is observed for the solution containing the PCR amplified APC gene fragment when compared to the control solution, which is characteristic of nanoparticle probe hybridization and aggregation as observed in previous systems (Storhoff et. al, J. Am. Chem. Soc. 1998, 120, 1959). The red shift leads to an increase in extinction for wavelengths above ˜550 nm, while it leads to a decrease in extinction for wavelength below ˜550 nm. Therefore, the colorimetric transition may be monitored as an increase or decrease in extinction at a number of wavelengths throughout the UV-visible spectrum, including 260 nm, 528 nm, or 570 nm. This data demonstrates that gold nanoparticle probes may be used to identify specific PCR amplified nucleic acid sequences using a simple spectrophotometric readout. A simplified detection system that monitors the extinction changes at specific wavelengths as shown in FIG. 8 could be applied to the real time detection of nucleic acid amplification when used in conjunction with BSA passivation as described in Example 3.

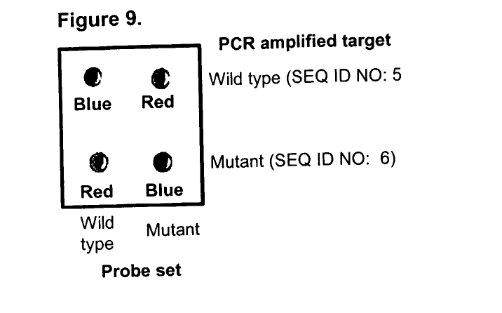

The next experiment was designed to demonstrate single base mismatch specificity of the gold probes, and also demonstrate that probe/target hybridization may be monitored by spotting an aliquot of the solution onto a membrane. Using both the wild type (SEQ ID NO: 1 and 3) and mutant (SEQ ID NO: 2 and 3) 30 nm gold probe sets, the wild type (SEQ ID NO: 5) and mutant (SEQ ID NO: 6) PCR amplicons could be distinguished on the basis of color by simply denaturing at 95° C., followed by hybridization at 4° C. for five minutes, and subsequently raising to a stringency temperature of 47.5° C. for an additional five minutes and spotting, FIG. 9. These initial results with 30 nm gold probes clearly demonstrate that sequence specific probe hybridization to PCR amplified sequences can be detected rapidly via a simple spotting onto a membrane.

What we claim:

1. A method for detecting the presence of a target polynucleotide in a sample comprising:
(a) providing a reaction and detection mixture comprising in combination:
(1) a sample;
(2) a nucleic acid amplification system; and
(3) a nanoparticle detection system comprising one or more types of nanoparticles having one or more types of oligonucleotides bound thereto, the oligonucleotides bound to the nanoparticles have a sequence that is complementary to at least a portion of the sequence of the amplified target polynucleotide;
(b) amplifying said target polynucleotide through at least one amplification cycle;
(c) allowing the binding of said oligonucleotides bound to the nanoparticle to said amplified target polynucleotide under conditions effective to allow hybridization between said oligonucleotides bound to the nanoparticle and said amplified target polynucleotide;
(d) determining the amount of signal generated as a result of the binding of the oligonucleotide bound to the nanoparticle to said amplified target polynucleotide;
(e) optionally repeating steps (b)-(d); and
(f) detecting the presence of said target polynucleotide by analyzing for the amount of signal produced after at least one amplification cycle.

2. The method according to claim 1 wherein said nanoparticle detection system comprises at least two or more types of nanoparticles having oligonucleotides bound thereto, at least some of the oligonucleotides in each type of nanoparticles have a sequence that can bind to different portions of the amplified target polynucleotide.

3. The method according to claim 1 wherein:
(a) the target polynucleotide comprises a first and a second complimentary strand; and
(b) the nucleic acid amplification system comprises:
(1) a thermostable DNA polymerase;
(2) 2′ deoxynucleoside-5′-triphosphates;
(3) a forward-primer capable of binding to the first complimentary strand; and
(4) a reverse-primer capable of binding to the second complimentary strand in a position that will direct DNA synthesis toward the site of annealing of the forward-priming oligonucleotide.

4. The method according to claim 1 wherein the nucleic acid amplification system is the polymerase chain reaction, nucleic acid sequence based amplification, transcription mediated amplification, or ligase chain reaction.

5. The method according to claim 4 wherein the nucleic acid amplification system is the polymerase chain reaction.

6. The method according to claim 5 wherein the amplification system further includes a thermal labile antibody against the thermal stable DNA polymerase.

7. The method according to claim 1 wherein said signal determinations are made during an exponential phase of the amplification process.

8. The method according to claim 1 wherein the method is used to determine the quantity of said target polynucleotide in a sample, said method further comprises:
(a) determining a threshold cycle number at which the signal generated from amplification of the target polynucleotide in a sample reaches a fixed threshold value above a baseline value; and
(b) calculating the quantity of the target polynucleotide in the sample by comparing the threshold cycle number determined for the target polynucleotide in a sample with the threshold cycle number determined for target polynucleotides of known amounts in standard solutions.

9. The method according to claim 1 wherein the signal is brought about by hybridization of the oligonucleotides on the nanoparticles with the amplified target polynucleotide.

10. The method according to claim 9 wherein the signal produced by the nanoparticles is an optical change.

11. The method according to claim 10 wherein the signal produced by the nanoparticles is a colorimetric change.

12. The method according to claim 1 wherein the conditions include freezing and thawing.

13. The method according to claim 1 wherein the conditions include heating and cooling.

14. The method according to claim 1 wherein the nanoparticles are made of a noble metal.

15. The method according to claim 14 wherein the nanoparticles are made of gold.

16. The method according to claim 1 wherein nanoparticle-labeled oligonucleotides are contacted with a protective agent.

17. The method according to claim 16 wherein the protective agent comprises albumin, casein, streptavidin, polyethylene glycol (PEG), gelatin, milk powder, an antibody, proteins, peptides, DNA, acid terminated and amine terminated thiols, detergents, an organic molecule having one or more thiol groups, or a polymer.

18. The method according to claim 17, wherein said acid terminated and amine terminated thiol comprise mercaptourdecanoic acid or mercaptoethylamine.

19. The method according to claim 17 wherein said organic molecule having one or more thiol groups comprises a thiol containing peptide.

20. The method according to claim 19 wherein said thiol containing peptide is glutathione.

21. The method according to claim 17 wherein said albumin is bovine serum albumin.

22. The method according to claim 17 wherein said polymer is an inorganic or organic polymer with affinity for the surface of a nanoparticle.

23. The method according to claim 17 wherein said gelatin is fish gelatin.

24. The method according to claim 17 wherein said detergent comprises sodium dodecyl sulfate or Tween 20.

25. A method for detecting the presence of a target polynucleotide in a sample, the target polynucleotide comprising a first and a second complimentary strand, said method comprising:
(a) providing a reaction and detection mixture comprising in combination:
(1) a sample,
(2) a thermostable DNA polymerase,
(3) 2′ deoxynucleoside-5′-triphosphates,
(4) a forward-primer capable of binding to the first complimentary strand,
(5) a reverse-primer capable of binding to the second complimentary strand in a position that will direct DNA synthesis toward the site of annealing of the forward-priming oligonucleotide, and
(6) a nanoparticle detection system comprising one or more types of nanoparticles having one or more types of oligonucleotides bound thereto, the oligonucleotides bound to the nanoparticles have a sequence that is complementary to at least a portion of the sequence of the amplified target polynucleotide;
(b) denaturing said target polynucleotide for an initial denaturation period;
(c) denaturing said target polynucleotide for a cycle denaturation period;
(d) incubating the reaction and detection mixture to allow binding of said nanoparticle-labeled oligonucleotides and said amplified target polynucleotide under conditions effective to allow hybridization between said oligonucleotides bound to the nanoparticle and said amplified target polynucleotide;
(e) determining the amount of signal generated by the binding of said nanoparticle-labeled oligonucleotide with said amplified target polynucleotide;
(f) annealing said forward priming and reverse priming oligonucleotides to the target polynucleotide;
(g) synthesizing polynucleotide strands complementary to said first and second complementary strands of said target polynucleotide, said synthesis being catalyzed by the thermostable DNA polymerase;
(h) optionally repeating steps (c)-(h); and
(i) detecting the presence of said target polynucleotide by analyzing the amount of signal generated after at least one amplification cycle.

26. A method for detecting the presence of a target polynucleotide in a sample, the target polynucleotide comprising a first and a second complimentary strand, said method comprising:
(a) providing a reaction and detection mixture comprising in combination:
(1) sample,
(2) a thermostable DNA polymerase,
(3) 2′ deoxynucleoside-5′-triphosphates,
(4) a forward-primer comprising a nanoparticle-labeled DNA primer sequence capable of binding to the first complimentary strand,
(5) a reverse-primer capable of binding to the second complimentary strand in a position that will direct DNA synthesis toward the site of annealing of the forward-priming oligonucleotide,
(6) a nanoparticle detection system comprising one or more types of nanoparticles having one or more types of oligonucleotides bound thereto, the oligonucleotides bound to the nanoparticles have a sequence that is complementary to at least a portion of the sequence of the extension product of the nanoparticle labeled DNA primer sequence, and
(b) denaturing said target polynucleotide for an initial denaturation period;
(c) denaturing said target polynucleotide for a cycle denaturation period;
(d) annealing said forward priming and reverse priming oligonucleotides to the target polynucleotide;
(e) determining the amount of signal generated by the binding of the extended DNA sequence bound through the nanoparticle labeled primer to the complementary nanoparticle probe and the nanoparticles having oligonucleotides bound thereto;
(g) synthesizing polynucleotide strands complementary to said first and second complementary strands of said target polynucleotide, said synthesis being catalyzed by the thermostable DNA polymerase;
(h) optionally repeating steps (c)-(h); and
(i) detecting the presence of said target polynucleotide by analyzing the amount of signal generated after at least one amplification cycle.

27. A method for detecting the presence of a target polynucleotide in a sample, the target polynucleotide comprising a first and a second complimentary strand, said method comprising:
(a) providing a reaction and detection mixture comprising in combination:
(1) sample,
(2) a thermostable DNA polymerase,
(3) 2′ deoxynucleoside-5′-triphosphates,
(4) a forward-primer capable of binding to the first complimentary strand,
(5) a reverse-primer comprising a nanoparticle-labeled DNA primer sequence capable of binding to the second complimentary strand in a position that will direct DNA synthesis toward the site of annealing of the forward-priming oligonucleotide,
(6) a nanoparticle detection system comprising one or more types of nanoparticles having one or more types of oligonucleotides bound thereto, the oligonucleotides bound to the nanoparticles have a sequence that is complementary to at least a portion of the sequence of the extension product of the nanoparticle labeled DNA primer sequence, and
(b) denaturing said target polynucleotide for an initial denaturation period;
(c) denaturing said target polynucleotide for a cycle denaturation period;
(d) annealing said forward priming and reverse priming oligonucleotides to the target polynucleotide;
(e) determining the amount of signal generated by the binding of the extended DNA sequence bound through the nanoparticle labeled primer to the complementary nanoparticle probe;
(g) synthesizing polynucleotide strands complementary to said first and second complementary strands of said target polynucleotide, said synthesis being catalyzed by the thermostable DNA polymerase;
(h) optionally repeating steps (c)-(h); and
(i) detecting the presence of said target polynucleotide by analyzing the amount of signal generated after at least one amplification cycle.

28. A method for detecting the presence of a target polynucleotide in a sample, the target polynucleotide comprising a first and a second complimentary strand, said method comprising:
(a) providing a reaction and detection mixture comprising in combination:
(1) sample,
(2) a thermostable DNA polymerase,
(3) 2′ deoxynucleoside-5′-triphosphates,
(4) a forward-primer comprising a nanoparticle-labeled DNA primer sequence capable of binding to the first complimentary strand,
(5) a reverse-primer comprising a nanoparticle-labeled DNA primer sequence capable of binding to the second complimentary strand in a position that will direct DNA synthesis toward the site of annealing of the forward-priming oligonucleotide, and
(b) denaturing said target polynucleotide for an initial denaturation period;
(c) denaturing said target polynucleotide for a cycle denaturation period;
(d) annealing said forward priming and reverse priming oligonucleotides to the target polynucleotide;
(e) determining the amount of signal generated by the binding of the amplified DNA sequences attached to the nanoparticle labeled primers;
(g) synthesizing polynucleotide strands complementary to said first and second complementary strands of said target polynucleotide, said synthesis being catalyzed by the thermostable DNA polymerase;
(h) optionally repeating steps (c)-(h); and
(i) detecting the presence of said target polynucleotide by analyzing the amount of signal generated after at least one amplification cycle.

29. The method according to any one of claim 25, 26, 27, or 28 wherein said nanoparticle detection system comprises at least two or more types of nanoparticles having oligonucleotides bound thereto, at least some of the oligonucleotides in each type of nanoparticles have a sequence that can bind to different portions of the amplified target polynucleotide.

30. The method according to any one of claim 25, 26, 27, or 28 wherein the nucleic acid amplification system is the polymerase chain reaction, nucleic acid sequence based amplification, transcription mediated amplification, or ligase chain reaction.

31. The method according to claim 30 wherein the nucleic acid amplification system is the polymerase chain reaction.

32. The method according to claim 31 wherein the amplification system further includes a thermal labile antibody against the thermal stable DNA polymerase.

33. The method according to any one of claims 25, 26, 27, or 28 wherein said signal determinations are made during an exponential phase of the amplification process.

34. The method according to any one of claims 25, 26, 27, or 28 wherein the method is used to determine the quantity of said target polynucleotide in a sample, said method further comprising:
(a) determining a threshold cycle number at which the signal generated from amplification of the target polynucleotide in a sample reaches a fixed threshold value above a baseline value;
(b) calculating the quantity of the target polynucleotide in the sample by comparing the threshold cycle number determined for the target polynucleotide in a sample with the threshold cycle number determined for target polynucleotides of known amounts in standard solutions.

35. The method according of any one of claims 25, 26, 27, or 28 wherein the signal is brought about by hybridization of the oligonucleotides on the nanoparticles with the amplified target polynucleotide.

36. The method according to claim 35 wherein the signal produced by the nanoparticles is an optical change.

37. The method according to claim 28 wherein the signal produced by the nanoparticles is a colorimetric change.

38. The method according of any one of claims 25, 26, 27, or 28 wherein the conditions include freezing and thawing.

39. The method according of any one of claims 25, 26, 27, or 28 wherein the conditions include heating and cooling.

40. The method according of any one of claims 25, 26, 27, or 28 wherein the nanoparticles are made of a noble metal.

41. The method according to claim 40 wherein the nanoparticles are made of gold.

42. The method according to any of of claims 25, 26, 27, or 28 wherein nanoparticle-labeled oligonucleotides are contacted with a protective agent.

43. The method according to claim 42 wherein the protective agent comprises albumin, casein, streptavidin, polyethylene glycol (PEG), gelatin, milk powder, an antibody, proteins, peptides, DNA, acid terminated and amine terminated thiols, detergents, an organic molecule having one or more thiol groups, or a polymer.

44. The method of claim 42, wherein said acid terminated and amine terminated thiol comprise mercaptourdecanoic acid or mercaptoethylamine.

45. The method of claim 42 wherein said organic molecule having one or more thiol groups comprises a thiol containing peptide.

46. The method of claim 45 wherein said thiol containing peptide is glutathione.

47. The method according to claim 43 wherein said albumin is bovine serum albumin.

48. The method according to claim 43 wherein said polymer is an inorganic or organic polymer with affinity for the surface of a nanoparticle.

49. The method according to claim 43 wherein said gelatin is fish gelatin.

50. The method according to claim 43 wherein said detergent comprises sodium dodecyl sulfate or Tween 20.

51. A method for detecting the presence of a target polynucleotide in a sample comprising:
(a) providing a reaction and detection mixture comprising in combination:
(1) a sample;
(2) a nucleic acid amplification system; and
(3) a nanoparticle detection system comprising one or more types of nanoparticles having one or more types of oligonucleotides bound thereto, the oligonucleotides bound to the nanoparticles have a sequence that is complementary to at least a portion of the sequence of the amplified target polynucleotide;
(b) amplifying said target polynucleotide through at least one amplification cycle;
(c) allowing the binding of said oligonucleotides bound to the nanoparticle to said amplified target polynucleotide under conditions effective to allow hybridization between said oligonucleotides bound to the nanoparticle and said amplified target polynucleotide;
(d) observing a detectable change.

52. The method according to claim 51 wherein the detectable change is brought about by hybridization of the oligonucleotides on the nanoparticles with the amplified target polynucleotide.

53. A method for detecting the presence of a target polynucleotide in a sample comprising:
(a) providing a reaction and detection mixture comprising in combination:
(1) a sample;
(2) a nucleic acid amplification system; and
(3) a nanoparticle detection system comprising one or more types of nanoparticles having one or more types of oligonucleotides bound thereto, the oligonucleotides bound to the nanoparticles have a sequence that is complementary to at least a portion of the sequence of the amplified target polynucleotide;
(b) amplifying said target polynucleotide through at least one amplification cycle;
(c) allowing the binding of said oligonucleotides bound to the nanoparticle to said amplified target polynucleotide under conditions effective to allow hybridization between said oligonucleotides bound to the nanoparticles and said amplified target polynucleotides;
(d) observing a detectable change resulting from the hybridization of the oligonucleotides on the nanoparticles with the amplified target polynucleotide.

54. The method according to any one of claims 51 or 53 wherein:
(a) the target polynucleotide comprises a first and a second complimentary strand; and
(b) the nucleic acid amplification system comprises:
(4) a thermostable DNA polymerase;
(5) 2′ deoxynucleoside-5′-triphosphates;
(6) a forward-primer capable of binding to the first complimentary strand; and
(4) a reverse-primer capable of binding to the second complimentary strand in a position that will direct DNA synthesis toward the site of annealing of the forward-priming oligonucleotide.

55. The method according to any one of claims 51 or 53 wherein the nucleic acid amplification system is the polymerase chain reaction, nucleic acid sequence based amplification, transcription mediated amplification, or ligase chain reaction.

56. The method according to any one of claim 55 wherein the nucleic acid amplification system is the polymerase chain reaction.

57. The method according to any one of claim 56 wherein the amplification system further includes a thermal labile antibody against the thermal stable DNA polymerase.

58. The method according to any one of claims 51 or 53 wherein said signal determinations are made during an exponential phase of the amplification process.

59. The method according to any one of claims 51 or 53 wherein said signal determinations are made at the completion of the amplification process.

60. The method according to any one of claim 51 or 53 wherein the method is used to determine the quantity of said target polynucleotide in a sample, said method further comprises:
(a) determining a threshold cycle number at which the signal generated from amplification of the target polynucleotide in a sample reaches a fixed threshold value above a baseline value; and
(b) calculating the quantity of the target polynucleotide in the sample by comparing the threshold cycle number determined for the target polynucleotide in a sample with the threshold cycle number determined for target polynucleotides of known amounts in standard solutions.

61. The method according to any one of claims 51 or 53 wherein the detectable change is brought about by hybridization of the oligonucleotides on the nanoparticles with the amplified target polynucleotide.

62. The method according to claim 61 wherein the detectable change is an optical change.

63. The method according to claim 61 wherein the detectable change is a colorimetric change.

64. The method according to claim 63 wherein the colorimetric change is observable on a solid surface.

65. The method according to any one of claims 51 or 53 wherein the conditions include freezing and thawing.

66. The method according to any one of claims 51 or 53 wherein the conditions include heating and cooling.

67. The method according to any one of claims 51 or 53 wherein the nanoparticles are made of a noble metal.

68. The method according to claim 67 wherein the nanoparticles are made of gold.

69. The method according to claim 51 or 53 wherein nanoparticle-labeled oligonucleotides are contacted with a protective agent.

70. The method according to claim 69 wherein the protective agent comprises albumin, casein, streptavidin, polyethylene glycol (PEG), gelatin, milk powder, an antibody, proteins, peptides, DNA, acid terminated and amine terminated thiols, detergents, an organic molecule having one or more thiol groups, or a polymer.

71. The method according to claim 70, wherein said acid terminated and amine terminated thiol comprise mercaptourdecanoic acid or mercaptoethylamine.

72. The method according to claim 70 wherein said organic molecule having one or more thiol groups comprises a thiol containing peptide.

73. The method according to claim 72 wherein said thiol containing peptide is glutathione.

74. The method according to claim 70 wherein said albumin is bovine serum albumin.

75. The method according to claim 70 wherein said polymer is an inorganic or organic polymer with affinity for the surface of a nanoparticle.

76. The method according to claim 70 wherein said gelatin is fish gelatin.

77. The method according to claim 70 wherein said detergent comprises sodium dodecyl sulfate or Tween 20.

78. A kit comprising:
(a) a nucleic acid amplification system; and
(b) a nanoparticle detection system comprising one or more types of nanoparticles having one or more types of oligonucleotides bound thereto, said nanoparticles produced by a process comprising contacting a nanoparticle having oligonucleotides bound thereto with a protective agent in aqueous solution in amounts sufficient to substantially prevent interference of said nucleic acid amplification reaction in the presence of said nanoparticle.

79. The kit according to claim 78 wherein the nucleic acid amplification system comprises a thermostable DNA polymerase, 2′ deoxynucleoside-5′-triphosphates and optional primers.

80. The kit of claim 78 wherein said nanoparticle detection system comprises at least two or more types of nanoparticles having oligonucleotides bound thereto, at least some of the oligonucleotides in each type of nanoparticles have a sequence that can bind to different portions of the amplified target polynucleotide.

81. A nanoparticle having oligonucleotides bound thereto for use as a detection probe in a nucleic acid amplification reaction, said nanoparticle produced by contacting a nanoparticle having oligonucleotides bound thereto with a protective agent in aqueous solution in amounts sufficient to substantially prevent interference of said nucleic acid amplification reaction in the presence of said nanoparticle.

82. The nanoparticle according to claim 61 wherein the nanoparticle-labeled oligonucleotides are contacted with a protective agent.

83. The nanoparticle according to claim 82 wherein the protective agent comprises albumin, casein, streptavidin, polyethylene glycol (PEG), gelatin, milk powder, an antibody, proteins, peptides, DNA, acid terminated and amine terminated thiols, detergents, an organic molecule having one or more thiol groups, or a polymer.

84. The nanoparticle according to claim 83, wherein said acid terminated and amine terminated thiol comprise mercaptourdecanoic acid or mercaptoethylamine.

85. The nanoparticle according to claim 83 wherein said organic molecule having one or more thiol groups comprises a thiol containing peptide.

86. The nanoparticle according to claim 85 wherein said thiol containing peptide is glutathione.

87. The nanoparticle according to claim 83 wherein said albumin is bovine serum albumin.

88. The nanoparticle according to claim 83 wherein said polymer is an inorganic or organic polymer with affinity for the surface of a nanoparticle.

89. The nanoparticle according to claim 83 wherein said gelatin is fish gelatin.

90. The nanoparticle according to claim 83 wherein said detergent comprises sodium dodecyl sulfate or Tween 20.

91. The nanoparticle according to claim 81 wherein said oligonucleotides bound to the nanoparticle thereto have a sequence that can bind to at least a portion of an amplified target polynucleotide.
